(19) United States
(12) Patent Application Publication
(10) Pub. No.: US 2011/0224128 A1
(43) Pub. Date: Sep. 15, 2011

(54) METHODS AND COMPOSITIONS FOR TREATMENT OF MUSCULAR DYSTROPHY

(76) Inventors: Anne Whalen, Medway, MA (US); Jane Staunton, Roslindale, MA (US); Janine Steiger, South Boston, MA (US); Amy B. Wilson, Marlborough, MA (US); Yang Wu, Reading, MA (US)

(21) Appl. No.: 12/759,268

(22) Filed: Apr. 13, 2010

Related U.S. Application Data
(60) Provisional application No. 61/168,774, filed on Apr. 13, 2009.

(51) Int. Cl. A61K 38/02; A61K 31/573; A61K 31/58; A61K 31/519; A61K 31/5513; A61K 31/445; A61K 31/407; A61K 31/436; A61K 31/437; A61K 31/47; A61K 31/52; A61K 31/4985; A61K 31/50; A61K 31/122; A61K 31/663; A61K 31/713; A61P 21/00

(52) U.S. Cl. 514/1.1; 514/171; 514/262.1; 514/218; 514/315; 514/411; 514/291; 514/293; 514/311; 514/263.4; 514/307; 514/249; 514/247; 514/678; 514/108; 514/44 A

(57) ABSTRACT

The invention features methods, compositions, and kits useful for the treatment of muscular dystrophy, e.g., Duchenne muscular dystrophy, in a patient.

This application claims priority under 35 U.S.C. §119 to U.S. Provisional Patent Application No. 61/168,774, which was filed on Apr. 13, 2009, and is herein incorporated by reference in its entirety.

### BACKGROUND OF THE INVENTION

The invention relates to the treatment of muscular dystrophy.

Muscular dystrophies (MD) are a group of genetic muscle diseases in which muscle fibers are unusually susceptible to damage. Muscles, primarily voluntary muscles, become progressively weaker. In the late stages of muscular dystrophy, fat and connective tissue often replace muscle fibers. Some types of muscular dystrophy affect heart muscles, other involuntary muscles, and other organs.

Two of the most common types of muscular dystrophy are Duchenne muscular dystrophy (DMD) and Becker muscular dystrophy (BMD). Children diagnosed with DMD, the more severe of these types, usually lose independent mobility by the late teen years and die from related complications by the age of twenty-five.

There is no cure for muscular dystrophy, and the efficacy of current therapies is severely limited. More effective treatments for muscular dystrophy are needed.

### SUMMARY OF THE INVENTION

Based on the results of two screens identifying combinations of compounds having (1) stimulatory activity toward an alpha 7 (α7) integrin promoter element, or (2) inhibitory activity toward NF-kappa B (NF-κB)-mediated gene activation, the present invention features methods, compositions, and kits for the treatment of muscular dystrophy.

Accordingly, in a first aspect, the invention features a method for treating a patient having muscular dystrophy by administering to the patient a pair of agents selected from the pairs of Table 1, or analogs thereof, in amounts that together are effective to treat the patient. Optionally, the method includes administering a third agent that is a corticosteroid.

TABLE 1
Combination agent pairs

| Combination Agent Pairs | Combination Agent Pairs |
|---|---|
| Dipyridamole and MBCQ | Dilazep dihydrochloride and Dexamethasone |
| Everolimus and N-(2-Aminoethyl)-5- | Etonogestrel and Dilazep |
| Isoquinolinesulfonamide | |
| Ethaverine and MBCQ | Prednisolone and Dilazep |
| EHNA and Everolimus | Prednisolone and Ergoloid |
| Everolimus and Fasudil | Mitoxantrone and Etonogestrel |
| Dipyridamole and Everolimus | Prednisolone and Ethaverine |
| Dilazep and MBCQ | Prednisolone and Dihydroergotamine |
| MBCQ and N1 N12-diethylspermine 4HCL | Trequinsin and Mitoxantrone |
| Berberine and Papaverine | NKH 477 and Ethaverine |
| Fasudil and LY 294002 | Ergoloid and Dexamethasone |
| Berberine and MBCQ | Tretinoin and Etonogestrel |
| Adefovir Dipivoxil and LY 294002 | Prednisolone and Bromocriptine |
| Antimycin A and MBCQ | NKH 477 and Mitoxantrone |
| 10-Hydroxycamptothecin and MBCQ | Prednisolone and Cilobradine |
| Berberine and Fasudil | Verapamil and Prednisolone |
| Dipyridamole and S-Petasin | MS-275 and Mitoxantrone |
| Everolimus and MBCQ | Mitoxantrone and Ethaverine |
| MS-275 and N-(2-Aminoethyl)-5-Isoquinolinesulfonamide | MS-275 and Dilazep |
| Idebenone and Tretinoin | Prednisolone and Dilazep |
| 10-Hydroxycamptothecin and Idebenone | Verapamil and Mitoxantrone |
| Adefovir Dipivoxil and MBCQ | Trequinsin and Dexamethasone |
| MBCQ and Simvastatin | Etonogestrel and Calcitriol |
| MBCQ and Suberoylanilide Hydroxamic Acid | NKH 477 and MS-275 |
| 2-(p-Hydroxyanilino)-4-(p-chlorophenyl) thiazole and | Ethaverine and Dexamethasone |
| Everolimus | |
| MBCQ and PDTC, NH4 | NKH 477 and Etonogestrel |
| MBCQ and Tretinoin | Mitoxantrone and Bromocriptine |
| Dilazep and Everolimus | Mitoxantrone and Dilazep |
| Deguelin and Fasudil | Prednisolone and Drotaverine |
| LY 294002 and Physostigmine | Mitoxantrone and Dihydroergotamine |
| 10-Hydroxycamptothecin and LY 294002 | Etonogestrel and Dihydroergotamine |
| Adefovir Dipivoxil and Physostigmine | Mitoxantrone and Calcitriol |
| Everolimus and Physostigmine | Mivacurium Chloride and Mitoxantrone |
| Ethaverine and Tadalafil | Etonogestrel and Demecarium Bromide |
| Deguelin and MBCQ | Prednisolone and NKH 477 |
| Deguelin and MS-275 | Mitoxantrone and Cilobradine |
| Idebenone and MS-275 | Mivacurium Chloride and Etonogestrel |
| EHNA and MBCQ | Prednisolone and Calcitriol |
| EHNA and Fumagillin | Mitoxantrone and Deflazacort |
| Deguelin and N-(2-Aminoethyl)-5- | Trequinsin and Prednisolone |
| Isoquinolinesulfonamide | |
| Fasudil and N-(2-Aminoethyl)-5-Isoquinolinesulfonamide | Sulforaphane and Dihydroergotamine |
| 10-Hydroxycamptothecin and EHNA | Trequinsin and Dilazep |
| MBCQ and Physostigmine | Verapamil and Dexamethasone |
| MBCQ and N-(2-Aminoethyl)-5-Isoquinolinesulfonamide | Mitoxantrone and Ergoloid |
| MBCQ and S-Petasin | Prednisolone and MS-275 |
| MBCQ and Pamidronate | Dilazep and Dexamethasone |
| Dipyridamole and Methyldopa | Dihydroergotamine and Dexamethasone |
| Fasudil and N1 N12-diethylspermine 4HCL | Prednisolone and Epiandrosterone |
| Everolimus and Tretinoin | Etonogestrel and Dilazep |
| Florfenicol and MBCQ | Etonogestrel and Ethaverine |
| Fumagillin and N1 N12-diethylspermine 4HCL | Procaterol and Etonogestrel |
| Fumagillin and N-(2-Aminoethyl)-5- | Prednisolone and Mitoxantrone |
| Isoquinolinesulfonamide | |
| N-(2-Aminoethyl)-5-Isoquinolinesulfonamide and | Trequinsin and MS-275 |
| N1 N12-diethylspermine 4HCL | |
| EHNA and N-(2-Aminoethyl)-5-Isoquinolinesulfonamide | Sulforaphane and Mitoxantrone |
| Physostigmine and Tretinoin | Trequinsin and NKH 477 |
| EHNA and Florfenicol | MBCQ and Etonogestrel |
| Dipyridamole and Fumagillin | Drotaverine and Dexamethasone |
| Deguelin and Droxidopa | Prednisolone and Mivacurium Chloride |
| Fumagillin and MBCQ | Efavirenz and Dilazep |
| Ethaverine and Fasudil | Mitoxantrone and MBCQ |
| Ethaverine and Everolimus | Levalbuterol and Dilazep |
| 10-Hydroxycamptothecin and Etazolate | Unithiol Monohydrate and Etonogestrel |
| N-(2-Aminoethyl)-5-Isoquinolinesulfonamide and | Verapamil and Etonogestrel |
| Pamidronate | |
| Deguelin and Simvastatin | MS-275 and Deflazacort |
| Deguelin and Everolimus | Vinburnine and Mitoxantrone |
| Ergoloid and Everolimus | Vitamin A Acetate and Mitoxantrone |
| Ethaverine and LY 294002 | Prednisolone and N-Methyl-Paroxetine |
| Ethaverine and MS-275 | NKH 477 and Dilazep |
| Physostigmine and Simvastatin | Tretinoin and MS-275 |
| Deguelin and LY 294002 | Mitoxantrone and Dexamethasone |
| Dilazep and Ergoloid | Dexamethasone and Bromocriptine |
| Dipyridamole and MS-275 | Procaterol and Mivacurium Chloride |
| 10-Hydroxycamptothecin and Dipyridamole | Trequinsin and Deflazacort |
| Idebenone and MBCQ | Prednisolone and Amoxapine |
| Berberine and N-(2-Aminoethyl)-5- | Etonogestrel and Bromocriptine |
| Isoquinolinesulfonamide | |
| N1 N12-diethylspermine 4HCL and Pamidronate | NKH 477 and Ergoloid |
| EHNA and Pamidronate | Dilazep and Dihydroergotamine |
| Droxidopa and Simvastatin | Mitoxantrone and Demecarium Bromide |
| Droxidopa and Tretinoin | NKH 477 and MBCQ |
| MS-275 and N1 N12-diethylspermine 4HCL | Epiandrosterone and Dexamethasone |
| LY 294002 and MBCQ | MBCQ and Ethaverine |
| Methyldopa and Tretinoin | Rosuvastatin calcium and Mitoxantrone |
| Donepezil and Tretinoin | Quinidine and Prednisolone |
| Berberine and Florfenicol | N-Methyl-Paroxetine and Mitoxantrone |
| Dopamine and MBCQ | Bromocriptine and Bethanechol Chloride |
| Levalbuterol and MBCQ | Prednisolone and MBCQ |
| Antimycin A and LY 294002 | Dexamethasone and Cilobradine |
| Dilazep and MS-275 | Otilonium Bromide and MBCQ |
| Alendronate and Ergoloid | Mitoxantrone and Drotaverine |
| LY 294002 and N-(2-Aminoethyl)-5- | Drotaverine and Calcitriol |
| Isoquinolinesulfonamide | |
| Berberine and Everolimus | Dilazep and Demecarium Bromide |
| Ergoloid and N-(2-Aminoethyl)-5- | Prednisolone and Piperacetazine |
| Isoquinolinesulfonamide | |
| Adefovir Dipivoxil and Fasudil | MS-275 and Dexamethasone |
| Ethaverine and N-(2-Aminoethyl)-5- | Trequinsin and Mivacurium Chloride |
| Isoquinolinesulfonamide | |
| Adefovir Dipivoxil and Droxidopa | NKH 477 and Calcitriol |
| Fasudil and Physostigmine | NKH 477 and Mivacurium Chloride |
| Ergoloid and Pamidronate | Etonogestrel and Bexarotene |
| Dipyridamole and N-(2-Aminoethyl)-5- | Salmeterol Xinafoate and MS-275 |
| Isoquinolinesulfonamide | |
| Antimycin A and MS-275 | MS-275 and Etonogestrel |
| EHNA and Idebenone | Vitamin A Acetate and Trequinsin |
| 10-Hydroxycamptothecin and Donepezil | Otilonium Bromide and Etonogestrel |
| EHNA and S-Petasin | Verapamil and NKH 477 |
| Physostigmine and S-Petasin | MS-275 and MBCQ |
| Dipyridamole and Florfenicol | Demecarium Bromide and Calcitriol |
| Berberine and Fumagillin | Mitoxantrone and Methoxsalen |
| Fasudil and MBCQ | NKH 477 and Dilazep |
| Fumagillin and Physostigmine | Vinburnine and Prednisolone |
| 10-Hydroxycamptothecin and Fumagillin | Dihydroergotamine and Calcitriol |
| Berberine and MS-275 | MS-275 and Ethaverine |
| Dipyridamole and Ergoloid | K-252a and Ethaverine |
| EHNA and MS-275 | Mivacurium Chloride and K-252a |
| Levalbuterol and Physostigmine | Procaterol and Dilazep |
| Adefovir Dipivoxil and Ergoloid | Tretinoin and Ethaverine |
| PDTC, NH4 and Physostigmine | Mivacurium Chloride and Calcitriol |
| Alendronate and N-(2-Aminoethyl)-5- | NKH 477 and Deflazacort |
| Isoquinolinesulfonamide | |
| Adefovir Dipivoxil and Fumagillin | Ergoloid and Calcitriol |
| EHNA and N1 N12-diethylspermine 4HCL | MS-275 and Dilazep |
| Ergoloid and Methyldopa | Dilazep and Calcitriol |
| Andrographis and Fumagillin | MBCQ and Deflazacort |
| Deguelin and Fumagillin | Dihydroergotamine and Deflazacort |
| EHNA and Ergoloid | Dilazep and Deflazacort |
| 10-Hydroxycamptothecin and Everolimus | Ergoloid and Deflazacort |
| Everolimus and Methyldopa | Ethaverine and Deflazacort |
| Methyldopa and Simvastatin | MBCQ and Dilazep |
| Everolimus and Florfenicol | Trequinsin and Dilazep |
| Berberine and Etazolate | Dilazep and Deflazacort |
| Droxidopa and Fasudil | Drotaverine and Deflazacort |
| Adefovir Dipivoxil and Tadalafil | Dipyridamole and Dexamethasone |
| Alendronate and MS-275 | Prednisolone and Dipyridamole |
| Adefovir Dipivoxil and Methyldopa | Dipyridamole and Deflazacort |
| Alendronate and Idebenone | Papaverine and Prednisolone |
| Berberine and S-Petasin | Dexamethasone and Papaverine |
| Ergoloid and Isoetharine | AL-438 and Dilazep |
| Adefovir Dipivoxil and Ethaverine | AL-438 and Papaverine |
| Fumagillin and PDTC, NH4 | AL-438 and Ergoloid |
| 10-Hydroxycamptothecin and Droxidopa | Dexamethasone and Zardaverine |
| Fasudil and Simvastatin | AL-438 and Dihydroergotamine |
| Physostigmine and Tadalafil | Deflazacort and Zardaverine |
| 10-Hydroxycamptothecin and Berberine | Prednisolone and Zardaverine |
| Methyldopa and N1 N12-diethylspermine 4HCL | Deflazacort and Papaverine |
| Florfenicol and S-Petasin | 2-(4-acetoxyphenyl)-2-chloro-N-methyl- |
| | ethylammonium chloride and Dilazep |
| EHNA and Physostigmine | Dipyridamole and Mitoxantrone |
| 10-Hydroxycamptothecin and Fasudil | AL-438 and Ethaverine |
| MBCQ and Tadalafil | Mitoxantrone and Papaverine |
| Berberine and Ethaverine | 2-(4-acetoxyphenyl)-2-chloro-N-methyl- |
| | ethylammonium chloride and Ergoloid |
| EHNA and Methyldopa | 2-(4-acetoxyphenyl)-2-chloro-N-methyl- |
| | ethylammonium chloride and Papaverine |
| Berberine and EHNA | Mitoxantrone and Tetrahydropapaveroline |
| Droxidopa and MBCQ | Dexamethasone and Tetrahydropapaveroline |
| 10-Hydroxycamptothecin and Pamidronate | Prednisolone and Tetrahydropapaveroline |
| Andrographis and MBCQ | Deflazacort and Tetrahydropapaveroline |
| MS-275 and PDTC, NH4 | 2-(4-acetoxyphenyl)-2-chloro-N-methyl- |
| | ethylammonium chloride and Ethaverine |
| Berberine and Ergoloid | 2-(4-acetoxyphenyl)-2-chloro-N-methyl- |
| | ethylammonium chloride and Dihydroergotamine |
| 10-Hydroxycamptothecin and Methyldopa | MBCQ and Physostigmine |
| 10-Hydroxycamptothecin and Florfenicol | Fasudil and Methyldopa |

The agents of the pair may be administered within 28 days, 21 days, 14 days, 10 days, 7 days, 3 days, 2 days, 24 hours, 12 hours, six hours, two hours, or one hour of each other, or substantially simultaneously. Agents may be administered by any acceptable route (e.g., by oral, systemic, parenteral, topical (e.g., ophthalmic, dermatologic), intravenous, inhalational, or intramuscular administration).

In certain embodiments of any of the above methods, the patient being treated has not been diagnosed with or does not suffer from a disease other than muscular dystrophy or its complications.

In another aspect, the invention features a composition that includes a pair of agents selected from the pairs of Table 1. In one embodiment, the composition optionally contains excipients, while the only active agents in the composition are the pair of agents from Table 1.

Desirably, in any of the compositions of the invention, the two agents are present in amounts that, when administered together to a patient having muscular dystrophy, are effective to treat the patient. The composition may be formulated, for example, for oral, systemic, parenteral, topical (e.g., ophthalmic, dermatologic), intravenous, inhalational, or intramuscular administration.

In another aspect, the invention features a kit including a pair of active agents selected from the pairs of Table 1 and instructions for administering the agent to a patient having muscular dystrophy. The two agents may be included together in a composition or may be formulated separately.

Related embodiments of the invention are kits including a first agent selected from the agents of a pair of Table 1 and instructions for administering the first agent and the second agent of the pair to a patient having muscular dystrophy.

Optionally, in any of the methods, compositions, and kits of the invention, a functional or structural analog (e.g., one described herein) of an agent listed in Table I may be employed instead of the agent listed in Table 1.

The methods, compositions, and kits of the invention may optionally include the use of a corticosteroid agent that is not the first agent, second agent, or analog thereof of the pair from Table 1.

In any aspect of the invention, the patient may be any animal, e.g., a human or other mammal.

Compounds useful in the invention include those described herein in any of their pharmaceutically acceptable forms, including isomers such as diastereomers and enantiomers, salts, solvates, and polymorphs thereof, as well as racemic mixtures. Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

By “agent” is meant a compound, e.g., dipyridamole, or mixture of compounds, e.g., ergoloid mesylates, having a pharmacological activity. The terms “agent,” “compound,” and “drug” are used interchangeably herein.

The term “muscular dystrophy” refers to a group of genetic diseases characterized by progressive degeneration of muscle tissue.

By “glucocorticoid” is meant a synthetic or natural steroid hormone that binds the glucocorticoid receptor, preferably with selectivity over the mineralocorticoid receptor.

By “ENT inhibitor” is meant an agent that inhibits the activity of an equilibrative nucleoside transporter, e.g., an equilibrative adenosine transporter, in vitro, in vivo, or both, by at least 5%, e.g., by 10%, 25%, 50%, 60%, 70%, 80%, 90%, or 95%.

By “PDE inhibitor” is meant an agent that inhibits a phosphodiesterase enzyme in vitro, in vivo, or both, by at least 5%, e.g., by 10%, 25%, 50%, 60%, 70%, 80%, 90%, or 95%. A PDE inhibitor may be selective for a particular type of PDE, e.g., PDE type V (PDE5).

By an “acetylcholinesterase inhibitor” is meant an agent that inhibits the activity of an acetylcholinesterase in vitro, in vivo, or both by at least 5%, e.g., by 10%, 25%, 50%, 60%, 70%, 80%, 90%, or 95%.

By a “Rho kinase inhibitor” is meant an agent that inhibits that activity of Rho kinase in vitro, in vivo, or both by at least 5%, e.g., by 10%, 25%, 50%, 60%, 70%, 80%, 90%, or 95%. In vitro kinase assays or cell-based bioassays, e.g., using fluorescence microscopy, may be used to detect and measure the Rho kinase inhibitory activity of an agent.

By an “mTOR inhibitor” is meant a compound that inhibits the activity of mTOR, also known as FK506 binding protein 12-rapamycin associated protein 1 (FRAP1), in vitro, in vivo, or both by at least 5%, e.g., by 10%, 25%, 50%, 60%, 70%, 80%, 90%, or 95%.

By a “calcium channel blocker” is meant an agent that directly or indirectly inhibits an activity of a calcium channel, e.g., current frequency, by at least 5%, e.g., by 10%, 25%, 50%, 60%, 70%, 80%, 90%, or 95%. Blocking (inhibitory) activity can be measured by methods known in the art.

By a “bisphosphonate” is meant one of a class of compounds that has two phosphate groups. The generic chemical structure of bisphosphonates is described herein. Many bisphosphonates can prevent the loss of bone mass or lower blood calcium when administered to a patient.

By a “CoQ10 analog” is meant an analog of coenzyme Q10, also known as ubiquinone.

By a “corticosteroid” is meant a natural or synthetic steroid hormone that binds either glucocorticoid receptors, mineralocorticoid receptors, or both.

By “patient” is meant any animal, e.g., a human.

To “treat” is meant to administer one or more agents to measurably slow, prevent, or reverse the progression of muscle weakness or other symptom associated with having muscular dystrophy. Desirably, the slowing of disease progression is at least by 10%, 20%, 30%, 40%, 50%, 60%, 70%, 80%, 90%, 95%, or 99%, or the reversal of disease progression is by at least 5%, 10%, 20%, 30%, 40%, 50%, 60%, 70%, 80%, 90%, 95%, or 99%. A slowing, preventing, or reversal of disease progression may be measured by comparing the average severity of symptoms in a group of patients treated by the methods of the invention with the average severity of symptoms in a matched group of patients not treated by the methods of the invention. Alternatively, in certain embodiments, treatment is observed by a trained physician or other person skilled in the art as an appreciable or substantial relief of symptoms in a patient with muscular dystrophy. Treatment may occur by any mechanism that prevents, slows, or reverses symptoms associated with muscular dystrophy, e.g., muscle weakness, difficulty walking, mental retardation, cardiopulmonary symptoms, or other symptom, e.g., one described herein.

By “an effective amount” is meant the amount of an agent, alone or in combination with another agent, required to treat a patient with muscular dystrophy (e.g., any type of MD described herein) in a clinically relevant manner. A sufficient amount of an active agent used to practice the present invention for therapeutic treatment of muscular dystrophy varies depending upon the manner of administration, the age, body weight, and general health of the patient. Ultimately, the prescriber will decide the appropriate amount and dosage regimen. In a combination therapy of the invention, the effective amount of an agent may less be than the effective amount if the agent were administered in a non-combinatorial (single-agent) therapy. Additionally, an effective amount may be an amount of an agent in a combination therapy of the invention that is safe and efficacious in the treatment of a patient having muscular dystrophy over each agent alone as determined and approved by a regulatory authority (such as the U.S. Food and Drug Administration).

By “more effective” is meant that a treatment exhibits greater efficacy, or is less toxic, safer, more convenient, or less expensive than another treatment with which it is being compared. Efficacy may be measured by a skilled practitioner using any standard method that is appropriate for a given indication.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that treats muscular dystrophy and that is formulated for administration by intravenous injection will differ from a low dosage of the same agent formulated for oral administration.

In the generic descriptions of compounds of this invention, the number of atoms of a particular type in a substituent group may be given as a range, e.g., an alkyl group containing from 1 to 4 carbon atoms or C₁₋₄ alkyl. Reference to such a range is intended to include specific references to groups having each of the integer number of atoms within the specified range. For example, an alkyl group from 1 to 4 carbon atoms includes each of C₁, C₂, C₃, and C₄. A C₁₋₁₂ heteroalkyl, for example, includes from 1 to 12 carbon atoms in addition to one or more heteroatoms. Other numbers of atoms and other types of atoms may be indicated in a similar manner. The term “lower,” when referring to a particular substituent group, e.g., “lower alkyl” or “lower alkoxy,” generally refers to groups containing 5, 4, or fewer carbon atoms.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 12 ring carbon atoms, inclusive. Exemplary cyclic groups include cyclopropyl, cyclobutyl, cyclopentyl, and cyclohexyl groups.

By “C₁₋₄ alkyl” is meant a branched or unbranched hydrocarbon group having from 1 to 4 carbon atoms. A C₁₋₄ alkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₁₋₄ alkyls include, without limitation, methyl, ethyl, n-propyl, isopropyl, cyclopropyl, cyclopropylmethyl, n-butyl, iso-butyl, sec-butyl, tert-butyl, and cyclobutyl.

By “C₂₋₄ alkenyl” is meant a branched or unbranched hydrocarbon group containing one or more double bonds and having from 2 to 4 carbon atoms. A C₂₋₄ alkenyl may optionally include monocyclic or polycyclic rings, in which each ring desirably has from three to six members. The C₂₋₄ alkenyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₂₋₄ alkenyls include, without limitation, vinyl, allyl, 2-cyclopropyl-1-ethenyl, 1-propenyl, 1-butenyl, 2-butenyl, 3-butenyl, 2-methyl-1-propenyl, and 2-methyl-2-propenyl.

By “C₂₋₄ alkynyl” is meant a branched or unbranched hydrocarbon group containing one or more triple bonds and having from 2 to 4 carbon atoms. A C₂₋₄ alkynyl may optionally include monocyclic, bicyclic, or tricyclic rings, in which each ring desirably has five or six members. The C₂₋₄ alkynyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. C₂₋₄ alkynyls include, without limitation, ethynyl, 1-propynyl, 2-propynyl, 1-butynyl, 2-butynyl, and 3-butynyl.

By “C₂₋₆ heterocyclyl” is meant a stable 5- to 7-membered monocyclic or 7- to 14-membered bicyclic heterocyclic ring which is saturated, partially unsaturated, or unsaturated (aromatic), and which consists of 2 to 6 carbon atoms and 1, 2, 3, or 4 heteroatoms independently selected from N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

By “C₇₋₁₄ alkaryl” is meant an alkyl substituted by an aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 3 to 10 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁₋₇ heteroalkyl” is meant a branched or unbranched alkyl, alkenyl, or alkynyl group having from 1 to 7 carbon atoms in addition to 1, 2, 3, or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups. Examples of C₁₋₇ heteroalkyls include, without limitation, methoxymethyl and ethoxyethyl.

By “halide” or “halogen” is meant bromine, chlorine, iodine, or fluorine.

By “fluoroalkyl” is meant an alkyl group that is substituted with a fluorine atom.

By “perfluoroalkyl” is meant an alkyl group consisting of only carbon and fluorine atoms.

By “carboxyalkyl” is meant a chemical moiety with the formula —(R)—COOH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “hydroxyalkyl” is meant a chemical moiety with the formula —(R)—OH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkoxy” is meant a chemical substituent of the formula —OR, wherein R is selected from C₁₋₇ alkyl, C₂₋₇ alkenyl, C₂₋₇ alkynyl, heterocyclyl, C₆₋₁₂ aryl, C₇₋₁₄ alkaryl, C₃₋₁₀ alkheterocyclyl, or C₁₋₇ heteroalkyl.

By “aryloxy” is meant a chemical substituent of the formula —OR, wherein R is a C₆₋₁₂ aryl group.

By “alkylthio” is meant a chemical substituent of the formula —SR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “arylthio” is meant a chemical substituent of the formula —SR, wherein R is a C₆₋₁₂ aryl group.

By “quaternary amino” is meant a chemical substituent of the formula —(R)—N(R′)(R″)(R′″)⁺, wherein R, R′, R″, and R′″ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl, heteroalkyl, heteroaryl, and/or aryl groups, resulting in a positive charge at the nitrogen atom.

Other features and advantages of the invention will be apparent from the following Detailed Description and the claims.

### DETAILED DESCRIPTION OF THE INVENTION

We have identified compounds that increase gene expression from the α7 integrin promoter and that diminish expression from an NF-κB responsive promoter in mammalian cells. Both of these effects (NF-κB inactivation and α7 integrin upregulation) predict a therapeutic benefit for the treatment of muscular dystrophy. Accordingly, the present invention provides compositions, methods, and kits useful for the treatment of muscular dystrophy. In certain embodiments, the muscular dystrophy is Duchenne muscular dystrophy (DMD) or Becker muscular dystrophy (BMD). Mechanisms of the invention may include decreasing inflammation and cell death in muscle and promoting adhesion of sarcolemmal membranes. Any other mechanism is also possible.

Compositions, methods, and kits of the invention can employ any pair of agents selected from Table 1. Optionally, in any aspect of the invention, a functional or structural analog of an agent of Table 1 (e.g., one described herein) may be employed instead of the agent listed in Table 1. In one particular example, a patient having muscular dystrophy is administered a combination of two agents listed in Table 1 within 24 hours of each other in amounts that together are effective to treat the patient having muscular dystrophy. An effective amount of one or both of the agents may be a low dosage relative the effective amount of the agent when administered singly for muscular dystrophy or for a different indication.

### Muscular Dystrophy

Muscular dystrophy comprises a family of at least nine genetic diseases of the muscles: DMD, BMD, Emery-Dreifuss muscular dystrophy (EDMD), limb-girdle muscular dystrophy (LGMD), facioscapulohumeral muscular dystrophy (FSHD), myotonic muscular dystrophy (MMD), oculopharyngeal muscular dystrophy (OPMD), distal muscular dystrophy (DD), and congenital muscular dystrophy (CMD). Symptoms of muscular dystrophy may include progressive muscle weakness, developmentally delayed walking, abnormal gait, apparent lack of coordination, frequent falling, difficulty standing up, lordosis, enlarged calves, fatigue after routine physical activity, difficulty swallowing, difficulty opening the eyes, learning disability, mental retardation, and cardiopulmonary complications.

Common forms of muscular dystrophy are DMD and BMD, both of which are linked to the X chromosome and associated with mutations in the dystrophin gene. DMD is more severe, usually presenting in childhood and resulting in death within two to three decades. In DMD, eventually all of the major muscles are affected, and lung capacity may decrease, resulting in an increased susceptibility to respiratory infections. Cardiac and respiratory failure is common in DMD patients. Other forms of muscular dystrophy may present in adults and progress more slowly.

### Agents of the Invention

In various embodiments of the present invention, particular therapeutic agents may be employed. Certain agents and exemplary analogs are discussed in greater detail below. It is to be understood that an analog of any agent of Table 1 can be used instead of the agent of Table 1 in the methods, compositions, and kits of the invention.

### Glucocorticoids

In certain embodiments, the methods, compositions, and kits of the invention employ predisolone or deflazacort. Analogs of prednisolone and deflazacort include their respective structural analogs and other glucocorticoids. 2-(4-acetoxyphenyl)-2-chloro-N-methylethylammonium chloride, a compound with glucocorticoid-like anti-inflammatory properties, is also considered herein to be an analog of prednisolone and deflazacort.

Prednisolone

Prednisolone is described in U.S. Pat. Nos. 2,837,464 and 3,134,718 and has the following structure:

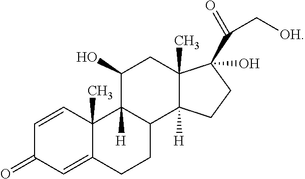

Analogs of prednisolone include 5-keto-4,5-seco-3-ynes of the estrane, androstane, and pregnane described in U.S. Pat. No. 3,835,160; the 17-benzoate of prednisolone described in U.S. Pat. No. 3,857,941; compounds of formula I in U.S. Pat. No. 7,498,321, e.g., 6α,9α-difluoro-11β-hydroxy-17α-[(isoxazole-5-carbonyl)oxy]-16α-methyl-3-oxo-androsta-1,4-diene-17β-carbothioic acid S-fluoromethyl ester; 17α-[(5-chlorothiophene-2-carbonyl)oxy]-6α,9α-difluoro-methyl ester; 11β-hydroxy-16α-methyl-3-oxo-androsta-1,4-diene-17β-carbothioic acid S-fluoromethyl ester; 6α,9α-difluoro-17α-[(3,5-dimethylisoxazole-4-carbonyl)oxy]-11β-hydroxy-16α-methyl-3-oxo-androsta-1,4-diene-17β-carbothioic acid S-fluoromethyl ester; 17α-[(5-chloro-4-methoxy-thiophene-3-carbonyl)oxy]-6α,9α-difluoro-1β-hydroxy-16α-methyl-3-oxo-androsta-1,4-diene-17β-carbothioic acid S-fluoromethyl ester; 6α,9α-difluoro-11β-hydroxy-16α-methyl-17α-[(4-methyl-1,2,3-thiadiazole-5-carbonyl)oxy]-3-oxo-androsta-1,4-diene-17β-carbothioic acid S-fluoromethyl ester; 17α-[(3-bromothiophene-2-carbonyl)oxy]-6α,9α-difluoro-1-1β-hydroxy-16α-methyl-3-oxo-androsta-1,4-diene-17β-carbothioic acid S-fluoromethyl ester; 17α-[(2,5-dichlorothiophene-3-carbonyl)oxy]-6α,9α-difluoro-11β-hydroxy-16α-methyl-3-oxo-androsta-1,4-diene-17β-carbothioic acid S-fluoromethyl ester; 17α-[(3-chlorothiophene-2-carbonyl)oxy]-6α,9α-difluoro-11β-hydroxy-16α-methyl-3-oxo-androsta-1,4-diene-17β-carbothioic acid S-fluoromethyl ester; 6α,9α-difluoro-11β-hydroxy-16α-methyl-17α-[(-5-methylisoxazole-4-carbonyl)oxy]-3-oxo-androsta-1,4-diene-17β-carbothioic acid S-fluoromethyl ester; 6α,9α-difluoro-11β-hydroxy-16α-methyl-17α-[(-1-methyl-1H-pyrrole-2-carbonyl)oxy]-3-oxo-androsta-1,4-diene-17β-carbothioic acid S-fluoromethyl ester; 6α,9α-difluoro-11β-hydroxy-16α-methyl-3-oxo-17α-[(1,3-thiazole-4-carbonyl)oxy]-androsta-1,4-diene-17β-carbothioic acid S-fluoromethyl ester; 6α,9α-difluoro-17α-[(2,4-dimethyl-1,3-thiazole-5-carbonyl)oxy]-11β-hydroxy-16α-methyl-3-oxo-androsta-1,4-diene-17β-carbothioic acid S-fluoromethyl ester; 6α,9α-difluoro-11β-hydroxy-16α-methyl-17α-[(-5-methylisoxazole-3-carbonyl)oxy]-3-oxo-androsta-1,4-diene-17β-carbothioic acid S-fluoromethyl ester; 6α,9α-difluoro-11β-hydroxy-16α-methyl-17α-[(3-methylisoxazole-5-carbonyl)oxy]-3-oxo-androsta-1,4-diene-17β-carbothioic acid S-fluoromethyl ester; 6α,9α-difluoro-17α-[(1,3-dimethyl-1H-pyrazole-5-carbonyl)oxy]-11β-hydroxy-16α-methyl-3-oxo-androsta-1,4-diene-17β-carbothioic acid S-fluoromethyl ester; 6α,9α-difluoro-11β-hydroxy-17α-[(isoxazole-3-carbonyl)oxy]-16α-methyl-3-oxo-androsta-1,4-diene-17β-carbothioic acid S-fluoromethyl ester; 6α,9α-difluoro-11β-hydroxy-17α-[(4-methoxy-thiophene-3-carbonyl)oxy]-16α-methyl-3-oxo-androsta-1,4-diene-17β-carbothioic acid S-fluoromethyl ester; 6α,9α-difluoro-11β-hydroxy-16α-methyl-17α-[(2-methyl-1,3-thiazole-4-carbonyl)oxy]-3-oxo-androsta-1,4-diene-17β-carbothioic acid S-fluoromethyl ester; 6α,9α-difluoro-17α-[(3-ethoxy-thiophene-2-carbonyl)oxy]-11β-hydroxy-16α-methyl-3-oxo-androsta-1,4-diene-17β-carbothioic acid S-fluoromethyl ester; 6α,9α-difluoro-11β-hydroxy-16α-methyl-3-oxo-17α-[(1,2,3-thiadiazole-4-carbonyl)oxy]-androsta-1,4-diene-17β-carbothioic acid S-fluoromethyl ester; 6α,9α-difluoro-11β-hydroxy-16α-methyl-3-oxo-17α-[(1H-pyrrole-2-carbonyl)oxy]-androsta-1,4-diene-17β-carbothioic acid S-fluoromethyl ester; 6α,9α-difluoro-11β-hydroxy-16α-methyl-3-oxo-17α-[(1,3-thiazole-5-carbonyl)oxy]-androsta-1,4-diene-17β-carbothioic acid S-fluoromethyl ester; 6α,9α-difluoro-11β-hydroxy-16α-methyl-3-oxo-17α-[(1,2,5-thiadiazole-3-carbonyl)oxy]-androsta-1,4-diene-17β-carbothioic acid S-fluoromethyl ester; 6α,9α-difluoro-11β-hydroxy-17α-[(isothiazole-3-carbonyl)oxy]-16α-methyl-3-oxo-androsta-1,4-diene-17β-carbothioic acid S-fluoromethyl ester; 6α,9α-difluoro-11β-hydroxy-17α-[(isothiazole-5-carbonyl)oxy]-16α-methyl-3-oxo-androsta-1,4-diene-17β-carbothioic acid S-fluoromethyl ester; 6α,9α-difluoro-11β-hydroxy-16α-methyl-17α-[(5-methylthiophene-2-carbonyl)oxy]-3-oxo-androsta-1,4-diene-17β-carbothioic acid S-fluoromethyl ester; 6α,9α-difluoro-11β-hydroxy-16α-methyl-17α-[(-3-methylthiophene-2-carbonyl)oxy]-3-oxo-androsta-1,4-diene-17β-carbothioic acid S-fluoromethyl ester; 6α,9α-difluoro-11β-hydroxy-16α-methyl-17α-[(-4-methyl-1,3-thiazole-5-carbonyl)oxy]-3-oxo-androsta-1,4-diene-17β-carbothioic acid S-fluoromethyl ester; 17α-[(1-ethyl-3-methyl-1H-pyrazole-5-carbonyl)oxy]-6α,9α-difluoro-11β-hydroxy-16α-methyl-3-oxo-androsta-1,4-diene-17β-carbothioic acid 5-fluoromethyl ester; 6α,9α-difluoro-17α-[(1-methyl-1H-imidazole-5-carbonyl)oxy]-11β-hydroxy-16α-methyl-3-oxo-androsta-1,4-diene-17β-carbothioic acid S-fluoromethyl ester; and 6α,9α-difluoro-11β-hydroxy-16α-methyl-3-oxo-17α-[(1,2,3-thiadiazole-5-carbonyl)oxy]-androsta-1,4-diene-17β-carbothioic acid S-fluoromethyl ester. Other exemplary analogs of prednisolone are described in U.S. Pat. Nos. 3,857,941, 3,956,349, 4,035,236, 4,041,055 and 5,225,335.

Deflazacort

Deflazacort is described in Belgian Patent No. 679,820, G. B. Patent No. 1,077,393, and U.S. Pat. No. 3,436,389. Deflazacort has the structure:

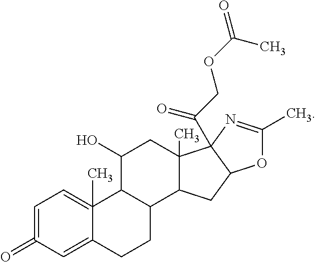

Analogs of deflazacort are described by the generic formula of U.S. Pat. No. 3,624,077, e.g., pregna-1,4-diene-11β,21-diol-3,20-dione-[17α,16α-d]-2′-phenyloxazoline 21-acetate; by formula I of U.S. Pat. No. 4,412,953, e.g., 3β-hydroxy-5-pregnen-20-one-[17α,16α-d]-2′-methyloxazoline; and by formula I of U.S. Pat. No. 4,440,764, e.g., 11β,21-dihydroxy-2′-methyl-5′H-pregna-1,4-dieno[17,16-d]-oxazole-3,20-dione-21-hemisuccinate.

Other exemplary glucocorticoids are dexamethasone, betamethasone, triamcinolone, triamcinolone acetonide, triamcinolone diacetate, triamcinolone hexacetonide, beclomethasone, dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, paramethasone acetate, methylprednisolone, methylprednisolone acetate, methylprednisolone sodium succinate, prednisolone acetate, prednisolone sodium phosphate, prednisolone tebutate, clocortolone pivalate, flucinolone, dexamethasone 21-acetate, betamethasone 17-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, clobetasol, cortisone, paramethasone, clocortolone, prednisolone 21-hemisuccinate free acid, prednisolone metasulphobenzoate, prednisolone terbutate, triamcinolone acetonide 21-palmitate, flurometholone, medrysone, loteprednol, fluazacort, betamethasone, prednisone, methylprednisolone, triamcinolone, hexacatonide, paramethasone acetate, diflorasone, fluocinolone and fluocinonide.

### Equilibrative Nucleoside Transporter Inhibitors

The compounds dipyridamole and dilazep are ENT inhibitors that may be used in the methods, compositions, and kits of the invention. Analogs of dipyridamole and dilazep include other ENT inhibitors, certain calcium channel blockers (e.g., nimodipine, nifedipine, nicardipine, nitrendipine, and felodipine, isradipine, and nioldipine), and structural analogs of dipyridamole and dialazep, e.g., nitrobenzylthioinosine, R75231, S6-(4-nitrobenzyl)-mercaptopurine riboside (NBMPR), and cannabinoids (e.g., cannabidiol; see Carrier et al. (Proc. Nat. Acad. Sci. USA 103 (20):7895-7900 (2006)). Other exemplary analogs of dipyrimadole and dilazep are described below.

Dipyridamole

Dipyridamole is an ENT inhibitor described in G.B. Patent No. 807,826 and U.S. Pat. No. 3,031,450. Dipyridamole has the structure:

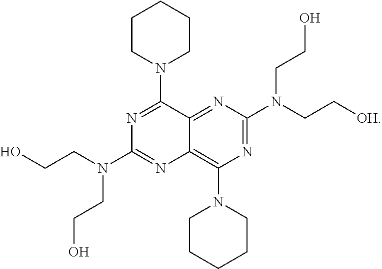

Certain dipyridamole analogs are described by formula (I) of U.S. Pat. No. 3,031,450:

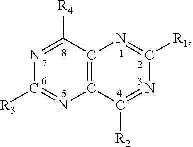

wherein two, three, or all four of the substituents R₁ through R₄ are basic groups, that is, primary, secondary, or tertiary amino groups; and, if only two or three of said substituents are basic groups, the remaining substituent or substituents are hydrogen, halogen, hydroxyl, mercapto, lower alkyl, phenyl, phenoxy, lower alkoxy, lower alkoxy-lower alkoxy, (di-lower alkyl-amino)-lower alkoxy, lower alkyl-mercapto, phenyl-mercapto, benzyl-mercapto, or carboxy-lower alkyl-mercapto.

Other analogs of dipyridamole are described by the chemical formula I of U.S. Pat. No. 3,687,950, e.g., 2-diethanol-amino-6-diethanolaminosulfonyl-4,8-dipiperidinopyrimido[5,4-d]pyrimidine, 2-diethanolamino-6-diethylaminoethylaminosulfonyl-4,8-dipiperidinopyrimido[5,4-d]pyrimidine, 2-diethanolamino16-morpholinosulfonyl-4,8-dipiperidinopyrimido[5,4-d]pyrimidine, 2-(N-methyl)ethanolamino-6-(N-methyl)ethanolaminosulfonyl-4,8-dipiperidinopyrimido[5,4-d]pyrimidine, 2-diethanolamino-6-dimethylaminosulfonyl-4,8-dipiperidinopyrimido[5,4-d]pyrimidine, 2-diethanolamino-6-(N-methyl)ethanolaminosulfonyl-4,8-dipiperidinopyrimido[5,4-d]pyrimidine, 2-diethanolaminosulfonyl-6-methylsulfonyl-4,8-dipiperidinopyrimido[5,4-d], 2,6-bis-(diethanolaminosulfonyl)-4,8-dipiperidinopyrimido[5,4-d]pyrimidine, and 2,6-bis(methylsulfonyl)-4,8-dipiperidinopyrimido[5,4-d]pyrimidine; by formula I of U.S. Pat. No. 4,478,833 e.g., 8-benzylthio-2-(2-hydroxyethyl-amino)-4-(1-oxido-thiomorpholino)-pyrimido[5,4-d]pyrimidine, 2-(2-hydroxyethyl-amino)-4-(1-oxido-thiomorpholino)-8-(L-1-phenylethylamino)-pyrimido[5,4-d]pyrimidine, and 8-benzylamino-2-(2-hydroxyethyl-amino)-4-(1-oxido-thiomorpholino)-pyrimido[5,4-d]pyrimidine; by formula I of U.S. Pat. No. 4,690,923, e.g., 4-(1-oxido-thiomorpholino)-8-(2-phenylethyl-mercapto)-2-piperazino-pyrimidino-[5,4-d]pyrimidine and 8-benzylthio-4-(1-oxido-thiomorpholino)-2-piperazino-pyrimido[5,4-d]pyrimidine; by formula I of U.S. Pat. No. 4,714,698, e.g., 1,8-methylthio-2-piperazino-4-pyrrolidino-pyrimido[5,4-d]pyrimidine; and by formula I of U.S. Pat. No. 4,963,541, e.g., 2,6-bis(2-(methylamino)ethanol)-4,8-bis(N-perhydroazocinyl)pyrimido[5,4-d]pyrimidine. Other analogs include mopidamol, BIBW 22, 2,6-bis(diethylamino)-4-piperidinopyrimido[5,4-d]pyrimidine (Mills et al. Biochem. J. 121:185 (1971)), RX-RA85, R-E 244 (4-(ethanolisopropanolamino)-2,7-di-(2′-methylmorpholino)-6-phenylpterine), 4-(1-oxidothiomorpholino)-8-phenethylthio-2-piperazino-pyrimido[5,4-d]pyrimidine, NU3026 (2,6-di-(2,2-dimethyl-1,3-dioxolan-4-yl)methoxy-4,8-di-piperidinopyrimidopyrimidine), NU3059 (2,6-bis-(2,3-dimethyoxypropoxy)-4,8-di-piperidinopyrimidopyrimidine), NU3060 (2,6-bis[N,N-di(2-methoxy)ethyl]-4,6-di-piperidinopyrimidopyrimidine), NU3076, NU3084, NU3108, and NU3121 (Smith et al. Clin. Cancer Res. 7:2105-2113 (2001)).

Dilazep

Dilazep is an ENT inhibitor described in G.B. Patent No. 1,107,470 and U.S. Pat. No. 3,532,685. Dilazep has the structure:

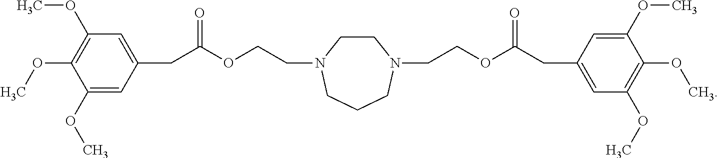

Exemplary analogs of dilazep are described by formula I of U.S. Pat. No. 4,035,494, e.g., 4′,4′″-[(N,N′-dimethyl-1,2-ethanediyldiimino)dimethylene]bis(pivalophenone)dihydrochloride, 4′,4′″-[(1,4-piperazinediyl)dimethylene]bis(pivalophenone), and 4′,4′″-[(1,4-homopiperazinediyl)dimethylene]bis(pivolophenone)dihydrochloride, and by formula I of U.S. Pat. No. 4,751,298, e.g., 1,4-bis-(3-hydroxypropyl)-1,4-diazepane dihydrochloride and 1,4-bis-[3-(3,4,5-trimethoxybenzoyloxy)propyl]-diazepane dihydrochloride. Other exemplary analogs are hexobendine, 3-[methyl-[2-[methyl-[3-(3,4,5-trimethoxybenzoyl)oxypropyl]amino]ethyl]amino]propyl 3,4,5-trimethoxybenzoate hydrochloride, 3-[methyl-[2-[methyl-[3-[(E)-3-(3,4,5-trimethoxyphenyl)prop-2-enoyl]oxypropyl]amino]ethyl]amino]propyl 3,4,5-trimethoxybenzoate, ST-7092, [(2S)-2-[methyl-[2-[methyl-[(2S)-1-(3,4,5-trimethoxybenzoyl)oxybutan-2-yl]amino]ethyl]amino]butyl] 3,4,5-trimethoxybenzoate, 3-[4-[3-[(E)-3-(3,4,5-trimethoxyphenyl)prop-2-enoyl]oxypropyl]piperazin-1-yl]propyl 3,4,5-trimethoxybenzoate, 3-[4-[3-(3,4,5-trimethoxybenzoyl)oxypropyl]-1,4-diazepan-1-yl]propyl 3-formyl-4,5-dimethoxybenzoate, LS-187114 ([(2S)-1-[methyl-[2-[methyl-[(2S)-2-(3,4,5-trimethoxybenzoyl)oxybutyl]amino]ethyl]amino]butan-2-yl]3,4,5-trimethoxybenzoate), buthobendin, LS-47408, KbioGR 000223 (7-(diethylamino)heptyl 3,4,5-trimethoxybenzoate), BRN 2708434 (3-[ethyl(2-phenylethyl)amino]propyl 3,4,5-trimethoxybenzoate), TMB-6 (6-(diethylamino)hexyl 3,4,5-trimethoxybenzoate), STK034691 (3-(dimethylamino)propyl 3,4,5-trimethoxybenzoate), BRN 2791312 (3-[ethyl-[1-(4-methoxyphenyl)propan-2-yl]amino]propyl 3,4,5-trimethoxybenzoate), STK182801 (1-(4-methylpiperazin-1-yl)propan-2-yl3,4,5-trimethoxybenzoate), BRN 2676106 (4-[ethyl(2-phenylethyl)amino]butyl 3,4,5-trimethoxybenzoate), BRN 2678814 (4-[2-phenylethyl(propyl)amino]butyl 3,4,5-trimethoxybenzoate), BRN 0867091 (2-[4-[(2E,6E)-3,7,11-trimethyldodeca-2,6,10-trienyl]piperazin-1-yl]ethyl 3,4,5-trimethoxybenzoate), BRN 2671634 (4-(diethylamino)butyl 3,4,5-trimethoxybenzoate), and 3-[4-(phenylmethyl)piperazin-1-yl]propyl 3,4,5-trimethoxybenzoate. Other analogs of dilazep include andiamine and K-7259 (Hogue et al., Pharmacology 277:207 (1996)).

### Acetylcholinesterase Inhibitors

In certain embodiments, the methods, compositions, and kits of the invention may employ the acetylcholinesterase inhibitor donepezil or physostigmine. Exemplary analogs of donepezil and physostigmine include their respective structural analogs. Other acetylcholinesterase inhibitors, including certain organophosphates (e.g., metrifonate), certain carbamates (e.g., physostigmine, neostigmine, pyridostigmine, and rivastigmine), certain phenanthrene derivatives (e.g., galantamine), certain piperidines (e.g., donepezil, also known as E2020), tacrine, ecothiopate, dyflos, ambenonium, demarcarium, and edrophonium, are also considered herein to be analogs of donepezil and physostigmine.

Donepezil

Donepezil has the structure:

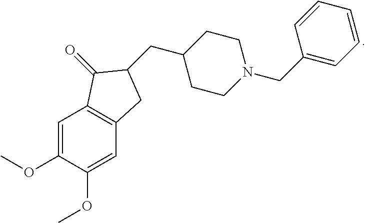

Exemplary analogs of donepezil are described in U.S. Pat. No. 7,105,540, e.g., 1-benzyl-4-(((5,6-dimethoxy-1-indanon)-2-yl)methylpiperidine, 1-benzyl-4-((5,6-dimethoxy-1-indanon)-2-ylidenyl)methylpiperidine, 1-benzyl-4-((5-methoxy-1-indanon)-2-yl)methylpiperidine, 1-benzyl-4-((5,6-diethoxy-1-indanon)-2-yl)methylpiperidine, 1-benzyl-4-((5,6-methylenedioxy-1-indanon)-2-yl)methylpiperidine, 1-(m-nitrobenzyl)-4-((5,6-dimethoxy-1-indanon)-2-yl)methylpiperidine, 1-cyclohexylmethyl-4-((5,6-dimethoxy-1-indanon)-2-yl)methylpiperidine, 1-(m-fluorobenzyl)-4-((5,6-dimethoxy-1-indanon)-2-yl)methylpiperidine, 1-benzyl-4-((5,6-dimethoxy-1-indanon)-2-yl)propylpiperidine, 1-benzyl-4-((5-isopropoxy-6-methoxy-1-indanon)-2-yl)methylpiperidine, and 1-benzyl-4-((5,6-dimethoxy-1-oxoindanon)-2-yl)propenylpiperidine.

Some analogs of donepezil are described by formula (II):

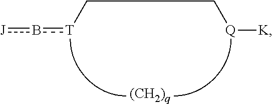

wherein J is (a) a group, substituted or unsubstituted, selected from the group consisting of (1) phenyl, (2) pyridyl, (3) pyrazyl, (4) quinolyl, (5) cyclohexyl, (6) quinoxalyl and (7) furyl; (b) a monovalent or divalent group, in which the phenyl may have a substituent(s), selected from the group consisting of (1) indanyl, (2) indanonyl, (3) indenyl, (4) indenonyl, (5) indanedionyl, (6) tetralonyl, (7) benzosuberonyl, (8) indanolyl and (9) C₆H₅—CO—CH(CH₃)—; (c) a monovalent group derived from a cyclic amide compound; (d) a lower alkyl or (e) a group of R²¹ —CH═CH— in which R²¹ is hydrogen or a lower alkoxycarbonyl; B is —(CHR²²)ᵣ—, —CO—(CHR²²)ᵣ—NR⁴—(CHR²²)ᵣ—, R⁴ being hydrogen, a lower alkyl, an acyl, a lower alkylsulfonyl, phenyl, a substituted phenyl, benzyl or a substituted benzyl, —CO—NR⁵—(CHR²²)ᵣ—, R⁵ being hydrogen, a lower alkyl or phenyl, —CH═CH—(CHR²²)ᵣ—, —OCOO—(CHR²²)ᵣ—, —OOC—NH—(CHR²²)ᵣ—, —NH—CO—(CHR²²)ᵣ—, —CH₂—CO—NH—(CHR²²)ᵣ—, —(CH₂)₂—NH—(CHR²²)ᵣ—, —CH(OH(CHR²²)ᵣ—, r being zero or an integer of 1 to 10, R²² being hydrogen or methyl so that one alkylene group may have no methyl branch or one or more methyl branch, ═(CH—CH═CH)_{b}—, b being an integer of 1 to 3, ═CH—(CH₂)^{c}—, c being zero or an integer of 1 to 9, ═(CH—CH)_{d}═, d being zero or an integer of 1 to 5; —CO—CH═CH—CH₂—, —CO—CH₂—CH(OH)—CH₂—, —CH(CH₃)—CO—NH—CH₂—, —CH═CH—CO—NH—(CH₂)₂—, —NH—, —O—, —S—, a dialkylaminoalkylcarbonyl or a lower alkoxycarbonyl; T is a nitrogen or carbon; Q is nitrogen, carbon or

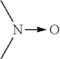

and q is an integer of 1 to 3; K is hydrogen, phenyl, a substituted phenyl, an arylalkyl in which the phenyl may have a substituent, cinnamyl, a lower alkyl, pyridylmethyl, a cycloalkylalkyl, adamantanemethyl, furylmenthyl, a cycloalkyl, a lower alkoxycarbonyl or an acyl; and [symbol] shows a single bond or a double bond, as described in U.S. Pat. No. 4,895,841, which is herein incorporated by reference.

Exemplary compounds of formula II are 1-benzyl-4-((5-methoxy-1-indanon)-2-yl)methylpiperidine, 1-benzyl-4-((5,6-diethoxy-1-indanon)-2-yl)methylpiperidine, 1-benzyl-4-((5,6-methylenedioxy-1-indanon)-2-yl)methylpiperidine, 1-(m-nitrobenzyl)-4-((5,6-dimethoxy-1-indanon)-2-yl)methylpiperidine, 1-(m-fluorobenzyl)-4-((5,6-dimethoxy-1-indanon)-2-yl)methylpiperidine, 1-benzyl-4-((5,6-dimethoxy-1-indanon)-2-yl)propylpiperidine, and 1-benzyl-4-((5-isopropoxy-6-methoxy-1-indanon)-2-yl)methylpiperidine.

Physostigmine

Physostigmine has the structure:

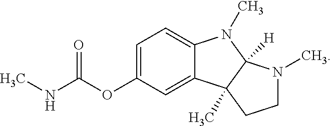

Exemplary physostigmine analogs are 1-desmethyl eserine, norphysostigmine, [(3aS,8bS)-3,4,8b-trimethyl-1,2,3,3a-tetrahydropyrrolo[2,3-b]indol-3-ium-7-yl]N-methylcarbamate, LS-190647 ([(3a,8b)-3,4,8b-trimethyl-2,3,3a,4-tetrahydro-1H-pyrrolo[2,3-b]indole-3,4-diium-7-yl]N-methylcarbamate), bisnorphysostigmine, eptastigmine, eseroline heptacarbamate, eseroline octylcarbamate, benzylnorphysostigmine, heptylstigmine, geneserine, eseroline, LS-139389 ([(3a,8b)-3,4,8b-trimethyl-2,3a-dihydro-1H-pyrrolo[2,3-b]indol-7-yl]N-octylcarbamate), MF247, MF 256, physostigmine analog 33, and N1,N8-bisbenzylnorphysostigmine.

Certain analogs of physostigmine are described by the formula (III):

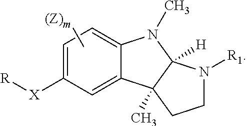

where (a) X is O or S; (b) R is H, loweralkyl,

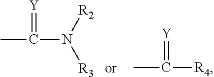

where Y is O or S; R₂ is alkyl, cycloalkyl, bicycloalkyl, cycloalkenyl, aryl, arylloweralkyl, heteroaryl or heteroarylloweralkyl, R₃ is H or alkyl, or the group —NR₂R₃ taken as a whole is 1-pyrrolidinyl, 1-piperidinyl, 4-morpholinyl, 4-thiomorpholinyl, 1-piperazinyl, 4-methyl-1-piperazinyl or 2-(2,6-dichlorophenylimino)-1-imidazolidinyl) and R₄ is hydrogen, loweralkyl, arylloweralkyl, diarylloweralkyl, aryl or heteroaryl; (c) m is 1 or 2; (d) each Z is independently H, loweralkyl, halogen, nitro, —NH₂, loweralkylcarbonylamino, arylcarbonylamino, loweralkoxycarbonylamino or loweralkylamino, and (e) R₁ is H, loweralkyl, arylloweralkyl, heteroarylloweralkyl, cycloalkylmethyl or loweralkenylmethyl, as described in U.S. Pat. Nos. 5,541,340, 4,900,748, 4,831,155, and 5,547,977, each of which is herein incorporated by reference. Exemplary compounds of formula III are (3aS-cis)-1,2,3,3a,8,8a-hexahydro-1,3a,8-trimethyl-pyrrolo[2,3-b]indol-5-ol, octadecyl carbamate ester; 7-chloro-(3aS-cis)-1,2,3,3a,8,8a-hexahydro-1,3a,8-trimethyl-pyrrolo[2,3-b]indol-5-ol, methyl carbamate ester; 7-bromo-(3aS-cis)-1,2,3,3a,8,8a-hexahydro-1,3a,8-trimethylpyrrolo[2,3-b]indol-5-ol, methyl carbamate ester; (3aS-cis)-1,2,3,3a,8,8a-hexahydro-1,3a,8-trimethyl-pyrrolo[2,3-b]indol-5-ol, N,N-diethyl carbamate ester; (3aS-cis)-1,2,3,3a,8,8a-hexahydro-1,3a,8-trimethyl-pyrrolo[2,3-b]indol-5-ol, cyclopentylmethyl carbamate ester; (3aS-cis)-1,2,3,3a,8,8a-hexahydro-1,3a,8-trimethyl-pyrrolo[2,3-b]indol-5-ol, (thien-3-yl)methyl carbamate ester; (3aS-cis)-1,2,3,3a,8,8a-hexahydro-1,3a,8-trimethyl-pyrrolo[2,3-b]indol-5-ol, benzyl carbamate ester; (3aS-cis)-1,2,3,3a,8,8a-hexahydro-1,3a,8-trimethyl-pyrrolo[2,3-b]indol-5-ol, (2-phenyl)ethyl carbamate ester; 3aS-[3aα,5(R*),8aα]]-1,2,3,3a,8,8a-hexahydro-1,3a,8-trimethyl-pyrrolo[2,3-b]indol-5-ol, (1-phenyl)ethyl carbamate ester; [3aS-[3aα,5(S*),8aα]]-1,2,3,3a,8,8a-hexahydro-1,3a,8-trimethylpyrrolo[2,3-b]indol-5-ol, (1-phenyl)ethyl carbamate ester, 7-chloro-[3aα,5(R*),8aα]-1,2,3,3a,8,8a-hexahydro-1,3a,8-trimethyl-pyrrolo[2,3-b]indol-5-ol, (1-phenyl)ethyl carbamate ester; 7-bromo-[3aα,5(R*),8aα]-1,2,3,3a,8,8a-hexahydro-1,3a,8-trimethyl-pyrrolo[2,3-b]indol-5-ol; (3aS-cis)-1,2,3,3a,8,8a-hexahydro-1,3a,8-trimethyl-pyrrolo[2,3-b]indol-5-ol, [1-(1-naphthyl)ethyl]carbamate ester; (3aS-cis)-1,2,3,3a,8,8a-hexahydro-1,3a,8-trimethyl-pyrrolo[2,3-b]indol-5-ol, cyclohexyl carbamate ester; 7-chloro-(3aS-cis)-1,2,3,3a,8,8a-hexahydro-1,3a,8-trimethyl-pyrrolo[2,3-b]indol-5-ol, cyclohexyl carbamate ester; 7-bromo-(3aS-cis)-1,2,3,3a,8,8a-hexahydro-1,3a,8-trimethyl-pyrrolo[2,3-b]indol-5-ol, cyclohexyl carbamate ester; (3aS-cis)-1,2,3,3a,8,8a-hexahydro-1,3a,8-trimethyl-pyrrolo[2,3-b]indol-5-ol, 4,4-dimethylcyclohexyl carbamate ester; (3aS-cis)-1,2,3,3a,8,8a-hexahydro-1,3a,8-trimethyl-pyrrolo[2,3-b]indol-5-ol, 4-ethylcyclohexyl carbamate ester; (3aS-cis)-1,2,3,3a,8,8a-hexahydro-1,3a,8-trimethyl-pyrrolo[2,3-b]indol-5-ol, spiro[5.5]undecan-3-yl carbamate ester; (3aS-cis)-1,2,3,3a,8,8a-hexahydro-1,3a,8-trimethyl-pyrrolo[2,3-b]indol-5-ol, cycloheptyl carbamate ester; (3aS-cis)-1,2,3,3a,8,8a-hexahydro-1,3a,8-trimethyl-pyrrolo[2,3-b]indol-5-ol, 1,2-dimethylcyclohexen-4-yl carbamate ester; (3aS-cis)-1,2,3,3a,8,8a-hexahydro-1,3a,8-trimethyl-pyrrolo[2,3-b]indol-5-ol, cyclohexen-1-yl carbamate ester; (3aS-cis)-1,2,3,3a,8,8a-hexahydro-1,3a,8-trimethyl-pyrrolo[2,3-b]indol-5-ol, bicyclo[2.2.1]heptan-2-yl carbamate ester; (3aS-cis)-1,2,3,3a,8,8a-hexahydro-1,3a,8-trimethyl-pyrrolo[2,3-b]indol-5-ol, 3-chlorophenyl carbamate ester; (3aS-cis)-1,2,3,3a,8,8a-hexahydro-1,3a,8-trimethyl-pyrrolo[[2,3-b]indol-5-ol, 4-chlorophenyl carbamate ester; (3aS-cis)-1,2,3,3a,8,8a-hexahydro-1,3a,8-trimethyl-pyrrolo[2,3-b]indol-5-ol, 2,6-dimethylphenyl carbamate ester; (3aS-cis)-1,2,3,3a,8,8a-hexahydro-1,3a,8-trimethyl-pyrrolo[2,3-b]indol-5-ol, 4-nitrophenyl carbamate ester; (3aS-cis)-1,2,3,3a,8,8a-hexahydro-1,3a,8-trimethyl-pyrrolo[2,3-b]indol-5-ol, 4-pyridinyl carbamate ester; (3aS-cis)-1,2,3,3a,8,8a-hexahydro-1,3a,8-trimethyl-pyrrolo[2,3-b]indol-5-ol, 4-methyl-piperazin-1-yl carbamate ester; (3aS-cis)-1,2,3,3a,8,8a-hexahydro-1,3a,8-trimethyl-pyrrolo[2,3-b]indol-5-ol, 4-morpholinyl carbamate ester; (3aS-cis)-1,2,3,3a,8,8a-hexahydro-1,3a,8-trimethyl-pyrrolo[2,3-b]indol-5-ol, 4-morpholinyl thiocarbamate ester; (3aS-cis)-1,2,3,3a,8,8a-hexahydro-1,3a,8-trimethyl-pyrrolo[2,3-b]indol-5-ol, 2-(2,6-dichlorophenylimino)-1-imidazolidinyl carbamate ester; (3aS-cis)-1,2,3,3a,8,8a-hexahydro-7-nitro-1,3a,8-trimethyl-pyrrolo[2,3-b]indol-5-ol, 3-chlorophenyl carbamate ester; 7-acetylamino-(3aS-cis)-1,2,3,3a,8,8a-hexahydro-1,3a,8-trimethylpyrrolo[2,3-b]indol-5-ol, 3-chlorophenyl carbamate ester; 6-bromo-(3aS-cis)-1,2,3,3a,8,8a-hexahydro-1,3a,8-trimethyl-pyrrolo[2,3-b]indol-5-ol, 3-chlorophenyl carbamate ester; 7-bromo-(3aS-cis)-1,2,3,3a,8,8a-hexahydro-1,3a,8-trimethyl-pyrrolo[2,3-b]indol-5-ol, n-heptyl carbamate ester; [3aS-[3aα,5(S*),8aα]]-1,2,3,3a,8,8a-hexahydro-7-nitro-1,3a,8-trimethylpyrrolo[2,3-b]indol-5-ol, (1-phenyl)ethyl carbamate ester; 7-bromo-(3aS-cis)-1,2,3,3a,8,8a-hexahydro-5-methoxy-1,3a,8-trimethylpyrrolo[2,3-b]indole; 7-chloro-(3aS-cis)-1,2,3,3a,8,8a-hexahydro-5-methoxy-1,3a,8-trimethylpyrrolo[2,3-b]indole; 7-acetylamino-(3aS-cis)-1,2,3,3a,8,8a-hexahydro-5-methoxy-1,3a,8-trimethylpyrrolo[2,3-b]indole; (3aS-cis)-1,2,3,3a,8,8a-hexahydro-5-methoxy-7-nitro-1,3a,8-trimethylpyrrolo[2,3-b]indole; 7-bromo-(3aS-cis)-1-cyclopropylmethyl-1,2,3,3a,8,8a-hexahydro-5-methoxy-3a,8-dimethylpyrrolo[2,3-b]indole; 7-bromo-(3aS-cis)-1,2,3,3a,8,8a-hexahydro-5-methoxy-1-(2-phenylethyl)-3a,8-dimethylpyrrolo[2,3-b]indole; 7-bromo-(3aS-cis)-1,2,3,3a,8,8a-hexahydro-5-methoxy-1-(2-propenyl)-3,8a-dimethylpyrrolo[2,3-b]indole; 7-bromo-1-(2-butenyl)-(3aS-cis)-1,2,3,3a,8,8a-hexahydro-5-methoxy-3,8a-dimethylpyrrolo[2,3-b]indole; 7-bromo-(3aS-cis)-1-cyclopropylmethyl-1,2,3,3a,8,8a-hexahydro-3a,8-dimethyl pyrrolo[2,3-b]indol-5-ol; 7-bromo-(3aS-cis)-1,2,3,3a,8,8a-hexahydro-1-(2-phenylethyl)-3a,8-dimethylpyrrolo[2,3-b]indol-5-ol; 7-bromo-(3aS-cis)-1,2,3,3a,8,8a-hexahydro-1-(2-propenyl)-3a,8-dimethylpyrrolo[2,3-b]indol-5-ol; 7-bromo-1-(2-butenyl)-(3aS-cis)-1,2,3,3a,8,8a-hexahydro-3a,8-dimethylpyrrolo[2,3-b]indol-5-ol; (3aS-cis)-1-cyclopropylmethyl-1,2,3,3a,8,8a-hexahydro-7-nitro-3a,8-dimethyl pyrrolo[2,3-b]indol-5-ol; (3aS-cis)-1,2,3,3a,8,8a-hexahydro-7-nitro-1-(2-propenyl)-3a,8-dimethylpyrrolo[2,3-b]indol-5-ol; 1-(2-butenyl)-(3aS-cis)-1,2,3,3a,8,8a-hexahydro-7-nitro-3a,8-dimethylpyrrolo[2,3-b]indol-5-ol; (3aS-cis)-1,2,3,3a,8,8a-hexahydro-7-nitro-1-(2-phenylethyl)-3a,8-dimethylpyrrolo[2,3-b]indol-5-ol; 7-bromo-(3a5-cis)-1,2,3,3a,8,8a-hexahydro-1,3a,8-trimethyl-pyrrolo[2,3-b]indol-5-ol acetate; (3aS-cis)-1,2,3,3a,8,8a-hexahydro-7-nitro-1,3a,8-trimethyl-pyrrolo[2,3-b]indol-5-ol acetate; (3aS-cis)-1,2,3,3a-8,8a-hexahydro-7-nitro-1,3a,8-trimethyl-pyrrolo[2,3-b]indol-5-ol trimethylacetate; 7-bromo-(3aS-cis)-1,2,3,3a,8,8a-hexahydro-1,3a,8-trimethyl-pyrrolo[2,3-b]indol-5-ol heptanoate; (3aS-cis)-1,2,3,3a,8,8a-hexahydro-7-nitro-1,3a,8-trimethyl-pyrrolo[2,3-b]indol-5-ol heptanoate; and 7-amino-(3aS-cis)-1,2,3,3a,8,8a-hexahydro-1,3a,8-trimethyl-pyrrolo[2,3-b]-5-ol, methyl carbamate ester.

### Rho Kinase Inhibitors

In certain embodiments, a Rho kinase inhibitor can be used in the compositions, methods, and kits of the invention. By a “Rho kinase inhibitor” is meant a compound that inhibits the activity of a Rho kinase by at least 5%, e.g., greater than 10%, 20%, 40%, 60%, 80%, 90%, or 95%. Inhibition of Rho kinase activity may be measured, e.g., by an in vitro assay with recombinant or purified Rho kinase, or by a cell-based reporter assay known in the art. Rho kinase inhibitors include fasudil, HA 1077 (Calbiochem), hydroxyfasudil, and Y-27632.

Fasudil

Fasudil is described in European Patent No. 187371 and U.S. Pat. No. 4,678,783 and has the following structure:

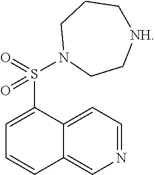

Certain analogs of fasudil are described by the formula (IV):

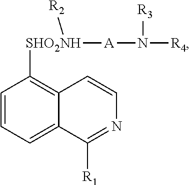

wherein R₁ represents a hydrogen atom, a chlorine atom or a hydroxyl group; and when R₁ represents a hydrogen atom, A represents an ethylene group unsubstituted or substituted with an alkyl group having 1 to 6 carbon atoms, a phenyl group or a benzyl group, R₂ and R₃ are directly bonded with each other, thereby forming a trimethylene group unsubstituted or substituted with an alkyl group having 1 to 6 carbon atoms, a phenyl group or a benzyl group, and R₄ represents a hydrogen atom or an alkyl group having 1 to 6 carbon atoms; and when R₁ represents a chlorine atom or a hydroxyl group, A represents an alkylene group having 2 to 6 carbon atoms, said group being unsubstituted or substituted with an alkyl group having 1 to 6 carbon atoms, R₂ and R₃ are not bonded with each other and each independently represent a hydrogen atom or an alkyl group having 1 to 6 carbon atoms, or R₂ and R₃ are directly bonded with each other, thereby forming an ethylene group unsubstituted or substituted with an alkyl group having 1 to 6 carbon atoms or a trimethylene group unsubstituted or substituted with an alkyl group having 1 to 6 carbon atoms, and R₄ represents a hydrogen atom, an alkyl group having 1 to 6 carbon atoms or an amidino group, as described in U.S. Pat. No. 4,678,783. Exemplary compounds of formula (IV) are 1-(5-isoquinolinesulfonyl)homopiperazine, 1-(5-isoquinolinesulfonyl)-2-methylhomopiperazine, 1-(5-isoquinolinesulfonyl)-3-methylhomopiperazine, 1-(5-isoquinolinesulfonyl)-6-methylhomopiperazine, 1-(5-isoquinolinesulfonyl)-2,3-dimethylhomopiperazine, 1-(5-isoquinolinesulfonyl)-3,3-dimethylhomopiperazine, 1-(5-isoquinolinesulfonyl)-3-ethylhomopiperazine, 1-(5-isoquinolinesulfonyl)-3-propylhomopiperazine, and 1-(5-isoquinolinesulfonyl)-3-isobutylhomopiperazine.

Other analogs of fasudil may be described by Formula I of U.S. Pat. No. 5,733,904; by Formulae II and IV of U.S. Pat. No. 4,798,897; by structural formula I in U.S. Pat. No. 4,857,301; by formula I of U.S. Pat. No. 5,081,246, e.g., N-[2-(4-benzyloxycarbonylpiperazinyl)-1-(p-methoxybenzyl)ethyl]-N-methyl-5-isoquinolinesulfonamide; by formula I of U.S. Pat. No. 5,244,895, e.g., N(1-(p-hydroxybenzyl)-2-(4-phenylpiperazinyl)ethyl)-5-isoquinoline sulfonamide, N-(2-(4-(m-chlorophenyl)piperazinyl)-1-(p-hydroxybenzyl)ethyl)-N-methyl-5-isoquinoline sulfonamide, N-(2-(4-benzyloxycarbonylpiperazinyl)-1-(p-hydroxybenzyl)ethyl)-N-methyl-5-isoquinoline sulfonamide, N-(2-(4-benzyloxycarbonylpiperazinyl)-1-(p-methoxybenzyl)ethyl)-N-methyl-5-isoquinoline sulfonamide, N-(p-hydroxybenzyl)-2-(4-phenylhomopiperazinyl)ethyl)-5-isoquinoline sulfonamide, N-(1-(p-hydroxybenzyl)-2-(4-(3-dichlorobenzyloxy)piperidino)ethyl)-5-isoquinoline sulfonamide, N-(1-(p-hydroxybenzyl)-2-(4-(3,4-dichlorobenzyloxy)piperidino)ethyl)-N-methyl-5-isoquinoline sulfonamide, N-(1-(p-methoxybenzyl)-2-(4-(3,4-dichlorobenzyloxy)piperidino)ethyl)-N-methyl-5-isoquinoline sulfonamide, N-(1-(p-hydroxybenzyl)-2-(4-phenylpiperidino)ethyl)-N-methyl-5-isoquinoline sulfonamide, N-(2-(4-benzyloxycarbonylhomopiperazinyl)-1-(p-hydroxybenzyl)ethyl)-N-methyl-5-isoquinoline sulfonamide, N-(2-(4-benzyloxycarbonylhomopiperazinyl)-1-(p-methoxybenzyl)ethyl)-N-(2-aminoethyl)-5-isoquinoline sulfonamide, and N-(2-(4-benzyloxycarbonylhomopiperazinyl)-1-(p-methoxybenzyl)ethyl)-N-(2-dimethylaminoethyl)-5-isoquinoline sulfonamide; by formula I of U.S. Pat. No. 5,245,034, e.g., N-anisyl-N-[2-(4-chlorocinnamylamino)ethyl]-5-isoquinolinesulfonamide; by formula I of U.S. Pat. No. 5,340,811, e.g., 1-(5-isoquinoline-sulfonylaminoethyl)-4-(3,4-methylenedioxybenzyl)piperazine; by formula I of U.S. Pat. No. 5,663,174; by Formula I of U.S. Pat. No. 5,747,507; by formula I of U.S. Pat. No. 5,942,505; and by formula I of U.S. Pat. No. 6,153,608, e.g., hexahydro-1-[(4-methyl-5-isoquinolinyl)sulfonyl]-1H-1,4-diazepine dihydrochloride, (S)-(+)-hexahydro-2-methyl-1-[(4-methyl-5-isoquinolinyl)sulfonyl]-1H-1,4-diazepine hydrochloride, hexahydro-7-methyl-1-[(4-methyl-5-isoquinolinyl)sulfonyl]-1H-1,4-diazepine dihydrochloride, hexahydro-5-methyl-1-[(4-methyl-5-isoquinolinyl)sulfonyl]-1H-1,4-diazepine dihydrochloride, hexahydro-2-methyl-1-[(4-methyl-5-isoquinolinyl)sulfonyl]-1H-1,4-diazepine hydrochloride, (R)-(−)-hexahydro-2-methyl-1-[(4-methyl-5-isoquinolinyl)sulfonyl]-1H-1,4-diazepine hydrochloride and (R)-(+)-hexahydro-5-methyl-1-[(4-methyl-5-isoquinolinyl)sulfonyl]-1H-1,4-diazepine hydrochloride.

### FKBP/mTOR Inhibitors

In certain embodiments of the methods, compositions, and kits of the invention, the FKBP/mTOR inhibitor everolimus or an everolimus analog may be employed. Analogs of everolimus include compounds structurally related to everolimus and other FKBP/mTOR inhibitors, e.g., temsirolimus, rapamycin, ascomycin, AP23573 (Ariad Pharmaceuticals), NVP-BEZ235, sirolimus, tacrolimus (FK 506), zotarolimus, and pimecrolimus.

Everolimus

Everolimus has the following structure:

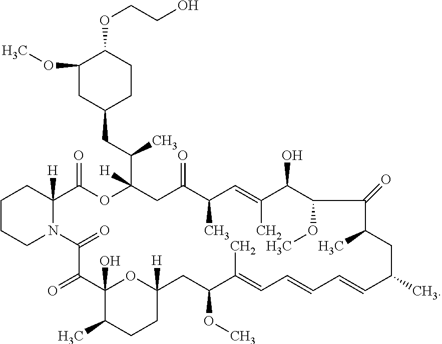

Exemplary analogs of everolimus are described by the general formula of U.S. Pat. No. 5,118,677, e.g., rapamycin-42-ester with 4-[[1-(4-chlorophenyl)methyl]amino]-4-oxobutanoic acid; by the general formula of U.S. Pat. No. 5,118,678, e.g., rapamycin 42-ester with (4-fluorophenyl)carbamic acid.; by formula I of U.S. Pat. No. 5,120,725, e.g., rapamycin-31,42-cyclic diester with hexanedioic acid; by formula I of U.S. Pat. No. 5,138,051, e.g., 33-Deoxy-33-hydroxyrapamycin; by the general structure of U.S. Pat. No. 5,194,447, e.g., rapamycin-31-ester with phenylsulfonylcarbamic acid; by the general structure of U.S. Pat. No. 5,378,836, e.g., rapamycin 27-oxime, 42-ester with 8-quinolinesulfonic acid; by the general structures of U.S. Pat. No. 5,387,680, e.g., C-22-methyl-rapamycin; and by formula I of U.S. Pat. No. 6,200,985, e.g., 16-pent-2-ynyloxy-32(S)-dihydro-rapamycin or 16-pent-2-ynyloxy-32(S)-dihydro-40-O-(2-hydroxyethyl)-rapamycin, 32-deoxo-rapamycin or 16-pent-2-ynyloxy-32-deoxo-rapamycin, 16-pent-2-ynyloxy-32(S)-dihydro-rapamycin or 16-pent-2-ynyloxy-32(S)-dihydro-40-O-(2-hydroxyethyl)-rapamycin, 32-deoxo-rapamycin or 16-pent-2-ynyloxy-32-deoxo-rapamycin, 16-pent-2-ynyloxy-32(S)-dihydro-rapamycin or 16-pent-2-ynyloxy-32(S)-dihydro-40-O-(2-hydroxyethyl)-rapamycin, 32-deoxo-rapamycin or 16-pent-2-ynyloxy-32-deoxo-rapamycin, 16-pent-2-ynyloxy-32(S)-dihydrorapamycin or 16-pent-2-ynyloxy-32(S)-dihydro-40-O-(2-hydroxyethyl)-rapamycin, 32-deoxo-rapamycin or 16-pent-2-ynyloxy-32-deoxo-rapamycin, 16-pent-2-ynyloxy-32(S)-dihydro-rapamycin or 16-pent-2-ynyloxy-32(S)-dihydro-40-O-(2-hydroxyethyl)-rapamycin, and 32-deoxo-rapamycin or 16-pent-2-ynyloxy-32-deoxo-rapamycin.

Certain everolimus analogs, e.g., pimecrolimus, are described by the formula I of U.S. Pat. No. 5,912,238. Other analogs of everolimus include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl rapamycin derivatives (U.S. Pat. No. 5,258,389); and deuterated rapamycin (U.S. Pat. No. 6,503,921). Yet other exemplary analogs of everolimus are described in U.S. Pat. Nos. 4,316,885, 5,023,262, 5,023,263, 5,023,264, 5,091,389, 5,202,332, and 5,169,851.

### Phosphodiesterase Inhibitors

The methods, compositions, and kits of the invention may employ ethaverine, drotaverine, papaverine, zardaverine, tetrahydropapaveroline, trequinsin, MBCQ (4-[[3,4-(methylenedioxy)benzyl]amino]-6-chloroquinazoline), or dipyridamole or an analog of one of these. Analogs of ethaverine, drotaverine, papaverine, zardaverine, tetrahydropapaveroline, trequinsin, MBCQ, or dipyridamole include their structural analogs and other phosphodiesterase (PDE) inhibitors.

Optionally, the PDE inhibitor selectively inhibits a particular type of PDE relative to other types. For example, a selective inhibitor may inhibit PDE type 5 (PDE5) at least 2-fold, 3-fold, 5-fold, 10-fold, 50-fold, or 100-fold more effectively than it inhibits another particular phosphodiesterase, e.g., PDE type II, type III, type IV, type VII, or type VIII, also known as PDE2, 3, 4, 7, and 8, respectively). A PDE inhibitor may also be non-selective or exhibit weak selectivity. Exemplary non-selective or weakly selective PDE inhibitors are theophylline, theobromine, IBMX, pentoxifylline and papaverine. The selectivity of a PDE inhibitor may be determined by measuring its IC₅₀ (the concentration required to achieve 50% inhibition of an enzyme) against at least two different phosphodiesterases.

In one embodiment of the invention, a PDE inhibitor selective for PDE5 is employed. Inhibitors of PDE5 may include griseolic acid derivatives, 2-phenylpurinones, phenylpyridones, fused and condensed pyrimidines, pyrimidopyrimidines, purine compounds, quinazoline compounds, phenylpyrimidinones, and imidazoquinoxalinones. Specific exemplary PDE5 inhibitors are dipyridamole, MBCQ, zaprinast, MY-5445, vinpocetine, FR229934, 1-methyl-3-isobutyl-8-methylamino)xanthine, IC-351, vardenafil, GF-196960, Sch-51866, and sodium-1-[6-chloro-4-(3,4-methylenedioxybenzyl)-aminoquinazolin-2-yl]piperidine-4-carboxylate sesquihydrate.

In preferred embodiments, a PDE inhibitor has an IC₅₀ of 100 μM or lower for a phosphodiesterase. In more preferred embodiments, the IC₅₀ of a phosphodiesterase inhibitor is 40, 20, or 10 μM or lower. In some embodiments, a phosphodiesterase inhibitor has an IC₅₀ of 40 μM, 20 μM, 10 μM, 5 μM, 1 μM, 100 nM, 10 nM, or lower for a particular type of phosphodiesterase. When a phosphodiesterase inhibitor is described herein as having activity against a particular type of phosphodiesterase, the inhibitor may also have activity against other types, unless otherwise stated.

Non-limiting examples of PDE inhibitors are theophylline(1,3-dimethylxanthine), caffeine, quercetin dihydrate, 4-(3-butoxy-4-methoxybenzyl)imidazolidin-2-one, propentofylline, 3-methyl-1-(5-oxohexyl)-7-propylxanthine), 3-isobutyl-1-methylxanthine, IBMX, 3-isobutyl-1-methyl-2,6(1H,3H)-purine-dione, 1-methyl-3-isobutylxanthine, 8-methoxymethyl-3-isobutyl-1-methylxanthine, enoximone, papaverine hydrochloride, calmidazolium chloride, imidazolium chloride, 1-[bis(4-chlorophenyl)methyl]-3-[2-(2,4-dichlorophenyl)-2-(2,4-dichlorobenzyloxy)ethyl]-1H-imidazolium chloride, SKF 94836, neuropeptide Y fragment 22-36, aminophylline hydrate, butein, etazolate hydrochloride, trifluoperazine dihydrochloride, and milrinone. Yet other examples are arofylline, atizoram, AWD-12-281 (N-(3,5-dichloro-4-pyridinyl)-2-[1-(4-fluorobenzyl)-5-hydroxy-1H-indol-3-yl]-2-oxoacetamide), BAY-19-8004 (ethanesulfonic acid 2-(2,4-dichlorophenylcarbonyl)-3-ureido-benzofuran-6-yl ester), benafentrine, CC-1088, CDC-801 (β-[3-(cyclopentyloxy)-4-methoxyphenyl]-1,3-dihydro-1,3-dioxo-2H-isoindole-2-propanamide), CDC-998, CI-1018, cilomilast(cis-[4-cyano-4-(3-cyclopentyloxy-4-methoxyphenyl)cyclohexane-1-carboxylic acid), cilostazol, cipamfylline(8-amino-1,3-bis(cyclopropylmethyl)xanthine), D-4396, D-4418 (N-(2,5-dichloro-3-pyridinyl)-8-methoxy-5-quinoline-carboxamide), darbufelone, denbufylline, ER-21355, filaminast, IC-485, indolidan, laprafylline, lixazinone, mesopram(5-(methoxy-3-propoxyphenyl)-5-methyl-2-oxazolidinone), nitraquazone, NM-702, olprinone, ORG-20241 (4-(3,4-dimethoxyphenyl)-N2-hydroxythiazole-2-carboxamidine), piclamilast, pumafentrine((−)-cis-9-ethoxy-8-methoxy-2-methyl-1,2,3,4,4a,10b-hexahydro-6-(4-diisopro-pylaminocarbonylphenyl)benzo[c][1,6]-naphthyridine), quazinone, RO-15-2041, roflumilast(3-(cyclopropl[methoxy)-N-(3,5-dichloro-4-pyridyl)-4-(difluoromethoxy)-benzamide), rolipram, SCH-351591, SH-636, tibenelast(5,6-diethoxybenzo[b]thiophene-2-carboxylic acid), tolafentrine, V-11294A (3-[[3-(cyclopentyloxy)-4-methoxyphenyl]methyl]-N-ethyl-8-(1-methylethyl)-3H-purin-6-amine), YM-58997 (4-(3-bromophenyl)-1-ethyl-7-methyl-1,8-naphthyridin-2(1H)-one), YM-976 (4-(3-chloro-phenyl)-1,7-diethylpyrido[2,3-d]pyrimidin-2(1H)-one), zardaverine, UK 66838, vasotrope, methyl 3-[6-(2H-3,4,5,6-tetrahydropyran-2-yloxy)-2-(3-thienylcarbonyl)benzo[β]furan-3-yl]propanoate, 4-[4-methoxy-3-(5-phenylpentyloxy)phenyl]-2-methylbenzoic acid, methyl 3-[2-[(4-chlorophenyl)carbonyl]-6-hydroxybenzo[β]furan-3-yl]propanoate, (R*,R*)-(±)-methyl 3-acetyl-4-[3-(cyclopentyloxy)-4-methoxyphenyl]-3-methyl-1-pyrrolidinecar-boxylate, and 4-(3-bromophenyl)-1-ethyl-7-methylhydropyridino[2,3-β]pyridin-2-one.

Additional examples of PDE inhibitors that may be useful in the compositions, methods, and kits provided herein are disclosed in U.S. Pat. No. 6,818,651, U.S. Pat. No. 6,737,436, U.S. Pat. No. 6,613,778, U.S. Pat. No. 6,617,357, U.S. Pat. No. 6,146,876, U.S. Pat. No. 6,838,559, U.S. Pat. No. 6,884,800, U.S. Pat. No. 6,716,987, U.S. Pat. No. 6,514,996, U.S. Pat. No. 6,740,655, U.S. Pat. No. 6,559,168, U.S. Pat. No. 6,069,151, U.S. Pat. No. 6,365,585, U.S. Pat. No. 6,313,116, U.S. Pat. No. 6,245,774, U.S. Pat. No. 6,011,037, U.S. Pat. No. U.S. Pat. No. 6,423,710, U.S. Pat. No. 6,372,777, U.S. Pat. No. 6,362,213, U.S. Pat. No. 6,313,156, U.S. Pat. No. 6,294,561, U.S. Pat. No. 6,258,843, U.S. Pat. No. 6,258,833, U.S. Pat. No. 6,043,263, U.S. Pat. No. 6,297,257, U.S. Pat. No. 6,251,923, U.S. Pat. No. 6,613,794, U.S. Pat. No. 6,407,108, U.S. Pat. No. 6,107,295, U.S. Pat. No. 6,103,718, U.S. Pat. No. 6,479,494, U.S. Pat. No. 6,545,158, U.S. Pat. No. 6,545,025, U.S. Pat. No. 6,498,160, U.S. Pat. No. 6,743,802, U.S. Pat. No. 6,787,554, U.S. Pat. No. 6,828,333, U.S. Pat. No. 6,869,945, U.S. Pat. No. 6,894,041, U.S. Pat. No. 6,924,292, U.S. Pat. No. 6,949,573, U.S. Pat. No. 6,953,810, U.S. Pat. No. 6,156,753, U.S. Pat. No. 5,972,927, U.S. Pat. No. 5,962,492, U.S. Pat. No. 5,814,651, U.S. Pat. No. 5,723,460, U.S. Pat. No. 5,716,967, U.S. Pat. No. 5,686,434, U.S. Pat. No. 5,502,072, U.S. Pat. No. 5,116,837, U.S. Pat. No. 5,091,431, U.S. Pat. No. 4,670,434, U.S. Pat. No. 4,490,371, U.S. Pat. No. 5,710,160, U.S. Pat. No. 5,710,170, U.S. Pat. No. 6,384,236, U.S. Pat. No. 3,941,785; in U.S. Patent publications 2005/0119225, 2005/0026913, 2005/0059686, 2004/0138279, 2005/0222138, 2004/0214843, 2004/0106631, 2003/0045557, 2002/0198198, 2003/0162802, 2003/0092908, 2003/0104974, 2003/0100571, 2003/0092721, and 2005/0148604; and in PCT publications WO 99/65880, WO 00/26201, WO 98/06704, WO 00/59890, WO9907704, WO9422852, WO 98/20007, WO 02/096423, WO 98/18796, WO 98/02440, WO 02/096463, WO 97/44337, WO 97/44036, and WO 97/44322.

Additional PDE inhibitors are shown in Table 2.

TABLE 2
Phosphodiesterase inhibitors

| | | PDE |
|---|---|---|
| | | Inhibitory |
| PDE inhibitor | Alternative Identifying Information | Activity |
| 349U85 | 6-piperidino-2(1H)-quinolinone | 3 |
| Adibendan | 5,7-dihydro-7,7-dimethyl-2-(4-pyridinyl)-pyrrolo(2,3-f)benzimidazol- | 3 |
| | 6(1H)-one | |
| Amlexanox | 2-amino-7-isopropyl-5-oxo-5H-[1]benzopyrano[2,3-b]pyridine-3- | 3, 4 |
| | carboxylic acid (U.S. Pat. No. 4,143,042) | |
| AM-230 | | |
| Amrinone | 5-amino-(3,4′-bipyridin)-6(1H)-one | 3, 4 |
| Anagrelide | U.S. Pat. No. 3,932,407 | 3, 4 |
| Apremilast | | 4 |
| AP 155 | 2-(1-piperazinyl)-4H-pyrido[1,2-a]pyrimidin-4-one | 4 |
| AR 12456 | CAS Reg. No. 100557-06-0 | 4 |
| Arofylline | 3-(4-chlorophenyl)-3,7-dihydro-1-propyl-1H-purine-2,6-dione | 4 |
| Ataquimast | 1-ethyl-3-(methylamino)-2(1H)-quinoxalinone | 3 |
| Atizoram | tetrahydro-5-[4-methoxy-3-[(1S,2S,4R)-2-norbornyloxy]phenyl]- | 4 |
| | 2(1H)-pyrimidinone | |
| ATZ 1993 | 3-carboxy-4,5-dihydro-1-[1-(3-ethoxyphenyl)propyl]-7-(5- | |
| | pyrimidinyl)methoxy-[1H]-benz[g]indazole (Teikoku Hormone) | |
| Avanafil | 4-[[(3-chloro-4-methoxyphenyl)methyl]amino]-2-[(2S)-2- | 5 |
| | (hydroxymethyl)pyrrolidin-1-yl]-N-(pyrimidin-2-ylmethyl)pyrimidine- | |
| | 5-carboxamide | |
| AVE 8112 | | 4 |
| AWD 12171 | | 5 |
| AWD 12187 | | 7 |
| AWD 12250 | | 5 |
| AWD12343 | | 4 |
| BAY 38-3045 | | 1 |
| BAY 60-7550 (Alexis | 2-(3,4-dimethoxybenzyl)-7-[(1R)-1-[(1R)-1-hydroxyethyl]-4-phenylbutyl]- | 2 |
| Biochemicals) | 5-methylimidazo[5,1-f][1,2,4]triazin-4(3H)-one | |
| BBB 022 | | 4 |
| Bemarinone | 5,6-dimethoxy-4-methyl-2(1H)-quinazolinone | 3 |
| Bemoradan | 6-(3,4-dihydo-3-oxo-1,4(2H)-benzoxazin-7-yl)-2,3,4,5-tetrahydro-5- | 3 |
| | methylpyridazin-3-one | |
| Benafentrine | (6-(p-acetamidophenyl)-1,2,3,4,4a,10b-hexahydro-8,9-dimethoxy-2- | 3, 4 |
| | methyl-benzo[c][1,6]naphthyridine | |
| BMY 20844 | 1,3-dihydro-7,8-dimethyl-2H-imidazo[4,5-b]quinolin-2-one | 4 |
| BMY 21190 | | 4 |
| BMY 43351 | 1-(cyclohexylmethyl)-4-(4-((2,3-dihydro-2-oxo-1H-imidazo(4,5- | 4 |
| | b)quinolin-7-yl)oxy)-1-oxobutyl)-piperazine | |
| BRL 50481 | 3-(N,N-dimethylsulfonamido)-4-methyl-nitrobenzene | 7 |
| C 3885 | | 4 |
| Caffeine citrate | 2-hydroxypropane-1,2,3-tricarboxylic acid | 4 |
| CC 10004 | N-(2-((1S)-1-(3-ethoxy-4-methoxyphenyl)-2-(methylsulfonyl)ethyl)-2,3- | 4 |
| | dihydro-1,3-dioxo-1H-isoindol-4-yl)-acetamide | |
| CC 1088 | | 4 |
| CC 3052 | See The Journal of Immunology, 161: 4236-4243 (1998). | 4 |
| CC 7085 | | 4 |
| CCT 62 | 6-[(3-methylene-2-oxo-5-phenyl-5-tetrahydrofuranyl)methoxy]quinolinone | 3 |
| CDC 998 | | 4 |
| CDP 840 | 4-((2R)-2-(3-(cyclopentyloxy)-4-methoxyphenyl)-2-phenylethyl)-pyridine | 4 |
| CGH 2466 | 2-amino-4-(3,4-dichlorophenyl)-5-pyridin-4-yl-thiazol | 4 |
| CI 1018 | N-(3,4,6,7-tetrahydro-9-methyl-4-oxo-1-phenylpyrrolo(3,2,1- | 4 |
| | jk)(1,4)benzodiazepin-3-yl)-4-pyridinecarboxamide | |
| CI 1044 | N-[9-amino-4-oxo-1-phenyl-3,4,6,7-tetrahydropyrrolo[3,2,1-jk][1,4]b- | 4 |
| | enzodiazepin-3(R)-yl]pyridine-3-carboxamide | |
| CI 930 | 4,5-dihydro-6-[4-(1H-imidazol-1-yl)phenyl]-5-methyl-3(2H)-pyridazinone | 3 |
| Cilomilast | 4-cyano-4-(3-cyclopentyloxy-4-methoxy-phenyl)cyclohexane-1-carboxylic | 4 |
| | acid (U.S. Pat. No. 5,552,438) | |
| Cilostamide | N-cyclohexyl-4-((1,2-dihydro-2-oxo-6-quinolinyl)oxy)-N-methyl- | 3 |
| | butanamide | |
| Cilostazol | 6-[4-(1-cyclohexyl-1H-tetrazol-5-yl)butoxy]-3,4-dihydro-2(1H)- | 3, 4 |
| | quinolinone (U.S. Pat. No. 4,277,479) | |
| Cipamfylline | 8-amino-1,3-bis(cyclopropylmethyl)-3,7-dihydro-1H-purine-2,6-dione | 4 |
| CK 3197 | 2H-imidazol-2-one, 1-benzoyl-5-(4-(4,5-dihydro-2-methyl-1H-imidazol-1- | |
| | yl)benzoyl)-4-ethyl-1,3-dihydro | |
| CP 146523 | 4′-methoxy-3-methyl-3′-(5-phenyl-pentyloxy)-biphenyl-4-carboxylic acid | 4 |
| CP 220629 | 1-cyclopentyl-3-ethyl-6-(2-methylphenyl)-7-oxo-4,5,6,7-tetrahydro-1H- | 4 |
| | pyrazolo[3,4-c]pyridine | |
| CP 248 | (Z)-5-fluoro-2-methyl-1-[p-(methylsulfonyl)benzylidene]indene-3-acetic | 2 |
| | acid | |
| CP 293121 | (S)-3-(3-cyclopentyloxy-4-methoxy)phenyl-2-isoxazoline-5-hydroxamic | 4 |
| | acid | |
| CP 353164 | 5-(3-cyclopentyloxy-4-methoxy-phenyl)-pyridine-2-carboxylic acid amide | 4 |
| CT 2820 | | |
| D 22888 | 8-methoxy-5-N-propyl-3-methyl-1-ethyl-imidazo [1,5-a]-pyrido [3,2-e]- | 4 |
| | pyrazinone | |
| D 4418 | N-(2,5-dichloro-3-pyridinyl)-8-methoxy-5-quinolinecarboxamide | 4 |
| Dasantafil | 7-(3-bromo-4-methoxyphenylmethyl)-1-ethyl-8-{[(1R,2R)-2- | 5 |
| | hydroxycyclopentyl] = amino}-3-(2-hydroxyethyl)-3,7-dihydro-1H-purine- | |
| | 2,6-dione | |
| Dipyridamole | 2-{[9-(bis(2-hydroxyethyl)amino)-2,7-bis(1-piperidyl)-3,5,8,10- | 5, 6, 7, 8, 10, |
| | tetrazabicyclo[4.4.0]deca-2,4,7,9,11-pentaen-4-yl]-(2- | 11 |
| | hydroxyethyl)amino}ethanol | |
| DN 9693 | 1,5-dihydro-7-(1-piperidinyl)-imidazo[2,1-b]quinazolin-2(3H)-one | 4 |
| | dihydrochloride hydrate | |
| Doxofylline | 7-(1,3-dioxolan-2-ylmethyl)-1,3-dimethyl-3,7-dihydro-1H-purine-2,6-dione | 4 |
| | (U.S. Pat. No. 4,187,308) | |
| E 4010 | 4-(3-chloro-4-metoxybenzyl)amino-1-(4-hydroxypiperidino)-6- | 5 |
| | phthalazinecarbonitrile monohydrochloride | |
| E 4021 | sodium 1-[6-chloro-4-(3,4-methylenedioxybenzyl)aminoquinazolin-2- | 4, 5 |
| | yl]piperidine-4-carboxylate sesquihydrate | |
| EHNA | erythro-9-(2-hydroxy-3-nonyl)adenine | 2, 3, 4 |
| EHT 0202 | 3,7-dimethyl-1-(5-oxohexyl)purine-2,6-dione | 4 |
| ELB 353 | | 4 |
| EMD 53998 | 5-(1-(3,4-dimethoxybenzoyl)-1,2,3,4-tetrahydro-6-quinolyl)-6-methyl-3,6- | 3 |
| | dihydro-2H-1,3,4-thiadiazin-2-one | |
| EMD 57033 | (+)-5-[1-(3,4-dimethoxybenzoyl)-3,4-dihydro-2H- | 3 |
| | quinolin-6-yl]-6-methyl-3,6-dihydro-1,3,4-thiadiazin-2-one | |
| EMD 57439 | (−)-5-[1-(3,4-dimethoxybenzoyl)-3,4-dihydro-2H- | 3 |
| | quinolin-6-yl]-6-methyl-3,6-dihydro-1,3,4-thiadiazin-2-one | |
| EMD 82639 | | 5 |
| EMR 62203 | | 5 |
| Enoximone | See U.S. Pat. No. 4,405,635. | 3 |
| Enprofylline | 3-propyl xanthine | 4 |
| ER 017996 | 4-((3,4-(methylenedioxy)benzy)amino)-6,7,8-trimethoxyquinazoline | |
| Etazolate | 1-ethyl-4-((1-methylethylidene)hydrazino)-1h-pyrazolo(3,4-b) pyridine-5- | 4 |
| | carboxylic acid | |
| Exisulind | (1Z)-5-fluoro-2-methyl-1-[[4-(methylsulfonyl)phenyl]methylene]-1H- | 2, 5 |
| | indene-3-acetic acid | |
| Filaminast | (1E)-1-(3-(cyclopentyloxy)-4-methoxyphenyl)-ethanone O- | 4, 7 |
| | (aminocarbonyl)oxime | |
| FR 226807 | N-(3,4-dimethoxybenzyl)-2-{[(1R)-2-hydroxy-1-methylethyl]amino}-5- | 5 |
| | nitrobenzamide | |
| FR 229934 | | 5 |
| GI 104313 | 6-{4-[N-[-2-[3-(2-cyanophenoxy)-2-hydroxypropylamino]-2- | 3 |
| | methylpropyl]carbamoylmethoxy-3-chlorophenyl]}-4,5-dihydro-3(2H) | |
| | pyridazinone | |
| GRC 3015 | | 4 |
| GSK 256066 | | 4 |
| GW 3600 | (7aS,7R)-7-(3-cyclopentyloxy-4-methoxyphenyl)-7a-methyl-2,5,6,7,7a- | 4 |
| | penta-hydro-2-azapyrrolizin-3-one | |
| GW 842470 | N-(3,5-dichloro-4-pyridinyl)-1-((4-fluorophenyl)methyl)-5-hydroxy-α-oxo- | 4 |
| | 1H-indole-3-acetamide | |
| Helenalin | CAS Reg. No. 6754-13-8 | 5 |
| Hydroxypumafentrine | | 4 |
| IBMX | 3-isobutyl-1-methylxanthine | 3, 4, 5 |
| Ibudilast | 1-(2-isopropyl-pyrazolo[1,5-a]pyridine-3-yl)-2-methylpropan-1-one (U.S. | 3, 4, 10, 11 |
| | Pat. No. 3,850,941) | (see Gibson et |
| | | al., Eur. J. |
| | | Pharm. 538: 39, |
| | | 2006) |
| IC 485 | | 4 |
| IPL 455903 | (3S,5S)-5-(3-cyclopentyloxy-4-methoxy-phenyl)-3-(3-methyl-benzyl)- | 4 |
| | piperidin-2-one | |
| Isbufylline | 1,3-dimethyl-7-isobutylxanthine | 4 |
| KF 17625 | 5-phenyl-1H-imidazo(4,5-c)(1,8)naphthyridin-4(5H)-one | 4 |
| KF 19514 | 5-phenyl-3-(3-pyridil) methyl-3H-imidazo[4,5-c][1,8]naphthyridin-4(5H)- | 1, 4 |
| | one | |
| KF 31327 | 3-ethyl-8-[2-[4-(hydroxymethyl)piperidin-1-yl]benzylamino]-2,3-dihydro- | 5 |
| | 1H-imidazo[4,5-g]quinazoline-2-thione | |
| Ks-505a | 1-carboxy-2,3,4,4a,4b,5,6,6a,6b,7,8,8a,8b,9,10,10a, | 1 |
| | 14,16,17,17a,17b,18,19,19a,19b,20,21,21a,21b,22,23,23a- | |
| | dotriacontahydro-14-hydroxy-8a,10a-bis(hydroxymethyl)-14-(3-methoxy- | |
| | 3-oxopropyl)-1,4,4a,6,6a,17b,19b,21b-octamethyl beta-D- | |
| | glucopyranosiduronic acid | |
| KT 734 | | 5 |
| KW 4490 | | 4 |
| L 686398 | 9-[1,S,2R)-2-fluoro-1-methylpropyl]-2-methoxy-6-(1-piperazinyl]-purine | 3, 4 |
| | hydrochloride | |
| L 826141 | 4-{2-(3,4-bis-difluromethoxyphenyl)-2-{4-(1,1,1,3,3,3-hexafluoro-2- | 4 |
| | hydroxypropan-2-yl)-phenyl]-ethyl}-3-methylpyridine-1-oxide | |
| L 869298 | (+)-1\|(S)-(+)-3-{2-[(3-cyclopropyloxy-4-difluromethoxy)-phenyl]-2-[5-(2- | 4 |
| | (1-hydroxy-1-trifluoromethyl-2,2,2-trifluoro)ethyl)-thiazolyl]ethyl}pyridine | |
| | N-oxide | |
| L-869299 | (−)-1\|(R)-(−)-3-{2-[(3-cyclopropyloxy-4-difluromethoxy)phenyl]-2-[5-(2- | 4 |
| | (1-hydroxy-1-trifluoromethyl-2,2,2-trifluoro)ethyl)thiazolyl]ethyl}pyridine | |
| | N-Oxide | |
| Laprafylline | 8-[2-[4-(dicyclohexylmethyl)piperazin-1-yl]ethyl]-1-methyl-3-(2- | 4 |
| | methylpropyl)-7H-purine-2,6-dione | |
| LAS 34179 | | 5 |
| LAS 37779 | | 4 |
| Levosimendan | See U.S. Pat. No. 5,569,657. | 3 |
| Lirimilast | methanesulfonic acid 2-(2,4-dichlorophenylcarbonyl)-3-ureidobenzo-furan- | 4 |
| | 6-yl ester | |
| Lixazinone | N-cyclohexyl-N-methyl-4-((1,2,3,5-tetrahydro-2-oxoimidazo(2,1- | 3, 4 |
| | b)quinazolin-7-yl)oxy)-butanamide | |
| LPDE4 inhibitor | Bayer | 4 |
| Macquarimicin A | See J Antibiot 48(6): 462-6, 1995. | |
| MEM 1414 | | 4 |
| MERCK1 | (5R)-6-(4-{[2-(3-iodobenzyl)-3-oxocyclohex-1-en-1-yl]amino}phenyl)-5- | 3 |
| | methyl-4,5-dihydropyridazin-3(2H)-one; dihydropyridazinone | |
| Mesopram | (5R)-5-(4-methoxy-3-propoxyphenyl)-5-methyl-2-oxazolidinone | 4 |
| Milrinone | 6-dihydro-2-methyl-6-oxo-3,4′-bipyridine)-5-carbonitrile (U.S. Pat. No. | 3, 4 |
| | 4,478,836) | |
| MIMX | 1 8-methoxymethyl-3-isobutyl-1-methylxantine | 1 |
| MN 001 | 4-[6-acetyl-3-[3-(4-acetyl-3-hydroxy-2-propylphenylthio)propoxy]-2- | 4 |
| | propylphenoxy]butyric acid | |
| Mopidamol | U.S. Pat. No. 3,322,755 | 4 |
| MS 857 | 4-acetyl-1-methyl-7-(4-pyridyl)-5,6,7,8-tetrahydro-3(2H)-isoquinolinone | 3 |
| Nanterinone | 6-(2,4-dimethyl-1H-imidazol-1-yl)-8-methyl-2(1H)-quinolinone | 3 |
| NCS 613 | See Boichot et al., J Pharmacol Exp Ther 292 (2): 647, 2000. | 4 |
| ND 1251 | | 4 |
| ND7001 | Neuro3D Pharmaceuticals | 2 |
| Nestifylline | 7-(1,3-dithiolan-2-ylmethyl)-1,3-dimethylpurine-2,6-dione | |
| NIK 616 | | 4 |
| NIP 520 | | 3 |
| NM 702 | | 5 |
| NSP 306 | | 3 |
| NSP 513 | | 3 |
| NSP 804 | 4,5-dihydro-6-[4-[(2-methyl-3-oxo-1-cyclopentenyl)-amino] phenyl]- | 3 |
| | 3(2H)-pyridazinone | |
| NSP 805 | 4,5-dihydro-5-methyl-6-[4-[(2-methyl-3-oxo-1-cyclopentenyl) | 3 |
| | amino]phenyl]-3(2H)-pyridazinone | |
| NVP ABE 171 | | 4 |
| Oglemilast | N-(3,5-dichloropyridin-4-yl)-4-difluoromethoxy-8- | 4 |
| | ((methylsulfonyl)amino)dibenzo(b,d)furan-1-carboxamide | |
| Olprinone | 5-imidazo[2,1-f]pyridin-6-yl-6-methyl-2-oxo-1H- | 3, 4 |
| | pyridine-3-carbonitrile | |
| ONO 1505 | 4-[2-(2-hydroxyethoxy)ethylamino]-2-(1H-imidazol-1-yl)-6-methoxy- | 5 |
| | quinazoline methanesulphonate | |
| ONO 6126 | | 4 |
| OPC 33509 | (−)-6-[3-[3-cyclopropyl-3-[(1R,2R)-2-hydroxyclohexyl]ureido]-propoxy]- | 3 |
| | 2(1H)-quinolinone | |
| OPC 33540 | 6-[3-[3-cyclooctyl-3-[(1R[*],2R[*])-2-hydroxycyclohexyl]ureido]- | 3 |
| | propoxy]-2(1H)-quinolinone | |
| ORG 20241 | N-hydroxy-4-(3,4-dimethoxyphenyl)-thiazole-2-carboximidamide | 3, 4 |
| ORG 30029 | N-hydroxy-5,6-dimethoxy-benzo[b]thiophene-2-carboximide | 3, 4 |
| | hydrochloride | |
| ORG 9731 | 4-fluoro-N-hydroxy-5,6-dimethoxy-benzo[b]thiophene-2- | 3, 4 |
| | carboximidamide methanesulphonate | |
| ORG 9935 | 4,5-dihydro-6-(5,6-dimethoxy-benzo[b]-thien-2-yl)-methyl-1-(2H)- | 3 |
| | pyridazinone | |
| OSI 461 | N-benzyl-2-[(3Z)-6-fluoro-2-methyl-3-(pyridin-4-ylmethylidene)inden-1- | 5 |
| | yl]acetamide hydrochloride | |
| Osthole | 7-methoxy-8-(3-methyl-2-butenyl)-2H-1-benzopyran-2-one | 5 |
| Ouazinone | (R)-6-chloro-1,5-dihydro-3-methyl-imidazo[2,1-b]quinazolin-2-one | 3 |
| PAB 13 | 6-bromo-8-(methylamino)imidazo[1,2-a]pyrazine | |
| PAB 15 | 6-bromo-8-(ethylamino)imidazo[1,2-a]pyrazine | |
| PAB 23 | 3-bromo-8-(methylamino)imidazo[1,2-a]pyrazine | |
| Papaverine | 1-[(3.4-dimethoxyphenyl)-methyl]-6,7-dimethoxyisoquinolone | 5, 6, 7, 10 |
| PDB 093 | | 4 |
| Pentoxifylline | 3,7-dimethyl-1-(5-oxohexyl)-3,7-dihydropurine-2,6-dione (U.S. Pat. No. | |
| | 3,422,107) | |
| Piclamilast | 3-cyclopentyloxy-N-(3,5-dichloropyridin-4-yl)-4-methoxy-benzamide | 4, 7 |
| Pimobendan | U.S. Pat. No. 4,361,563 | 3, 4 |
| Piroximone | 4-ethyl-1,3-dihydro-5-(4-pyridinylcarbonyl)-2H-imidazol-2-one | 3 |
| Prinoxodan | 6-(3,4-dihydro-3-methyl-2-oxoquinazolinyl)-4,5-dihydro-3-pyridazinone | |
| Propentofylline | U.S. Pat. No. 4,289,776 | 5 |
| Pumafentrine | rel-(M)-4-((4aR,10bS)-9-ethoxy-1,2,3,4,4a,10b-hexahydro-8-methoxy-2- | 4 |
| | methylbenzo(c)(1,6)naphthyridin-6-yl)-N,N-bis(1-methylethyl)-benzamide | |
| R 79595 | N-cyclohexyl-N-methyl-2-[[[phenyl (1,2,3,5-tetrahydro-2 oxoimidazo [2,1- | 3 |
| | b]-quinazolin-7-yl) methylene] amin] oxy] acetamide | |
| Revizinone | (E)—N-cyclohexyl-N-methyl-2-(((phenyl(1,2,3,5-tetrahydro-2- | 3 |
| | oxoimidazo(2,1-b)quinazolin-7-yl)methylene)amino)oxy)-acetamide | |
| Ro20-1724 | 4-(3-butoxy-4-methoxybenzyl)-2-imidazolidinone | 4 |
| Roflumilast | 3-(cyclopropylmethoxy)-N-(3,5-dichloro-4-pyridinyl)-4- | 4, 5 |
| | (difluoromethoxy)-benzamide | |
| Rolipram | 4-(3-cyclopentyloxy-4-methoxyphenyl)-2-pyrrolidone (U.S. Pat. No. | 4 |
| | 4,193,926) | |
| RPL554 | 9,10-dimethoxy-2(2,4,6-trimethylphenylimino)-3-(N-carbamoyl-2- | 3, 4 |
| | aminoethyl)-3,4,6,7-tetrahydro-2H-pyrimido[6,1-a]isoquinolin-4-one | |
| RPL565 | 6,7-dihydro-2-(2,6-diisopropylphenoxy)-9,10-dimethoxy-4H-pyrimido[6,1- | 3, 4 |
| | a]isoquinolin-4-one | |
| RPR 132294 | | 4 |
| RPR 132703 | | 4 |
| Saterinone | 1,2-dihydro-5-(4-(2-hydroxy-3-(4-(2-methoxyphenyl)-1- | 3 |
| | piperazinyl)propoxy)phenyl)-6-methyl-2-oxo-3-pyridinecarbonitrile | |
| Satigrel | 4-cyano-5,5-bis(4-methoxyphenyl)-4-pentenoic acid (U.S. Pat. No. | 2, 3, 5 |
| | 4,978,767) | |
| SCA 40 | 6-bromo-8-methylaminoimidazol[1,2-a]pyrazine-2carbonitrile | 3 |
| SCH 351591 | N-(3,5-dichloro-1-oxido-4-pyridinyl)-8-methoxy-2-(trifluoromethyl)-5- | 4 |
| | quinoline carboxamide | |
| SCH 45752 | J Antibiot (Tokyo). 1993 Feb; 46(2): 207-13 | |
| SCH 46642 | | 5 |
| SCH 51866 | cis-5,6a,7,8,9,9a-hexahydro-2-(4-(trifluoromethyl)phenylmethyl)-5-methyl- | 1, 5 |
| | cyclopent (4,5)imidazo(2,1-b)purin-4(3H)-one | |
| SCH 51866 | cis-5,6a,7,8,9,9a-hexahydro-2-[4-(trifluoromethyl)phenylmethyl]-5-methyl- | 1, 5 |
| | cyclopent[4,5]imidazo[2,1-b]purin-4(3H)-one | |
| SCH 59498 | cis-2-hexyl-5-methyl-3,4,5,6a,7,8,9,9a-octahydrocyclopent[4,5]imidazo- | 5 |
| | [2,-1-b]purin-4-one | |
| SDZ ISQ 844 | 6,7-dimethoxy-1-(3,4-dimethoxyphenyl)-3-hydroxymethyl-3,4- | 3, 4 |
| | dihydroisoquinoline | |
| SDZ MKS 492 | R(+)-(8-[(1-(3,4-dimethoxyphenyl)-2-hydroxyethyl)amino]-3,7-dihydro-7- | 3 |
| | (2-methoxyethyl)-1,3-dimethyl-1H-purine-2,6-dione | |
| Senazodan | | 3 |
| Siguazodan | N-cyano-N′-methyl-N″-[4-(1,4,5,6-tetrahydro-4-methyl-6-oxo-3- | 3, 4 |
| | pyridazinyl)phenyl]guanidine | |
| Sildenafil | 5-[2-ethoxy-5-(4-methyl-1-piperazinylsulfonyl)phenyl]-1-methyl-3-n- | 5 |
| | propyl-1,6-dihydro-7H-pyrazolo[4,3-d]pyrimidin-7-one (U.S. Pat. No. | |
| | 5,250,534) | |
| SK 3530 | | 5 |
| SKF 94120 | 5-(4-acetamidophenyl)pyrazin-2(1H)-one | 3 |
| SKF 95654 | ±-5-methyl-6-[4-(4-oxo-1,4-dihydropyridin-1-yl)phenyl]-4,5-dihydro- | 3 |
| | 3(2H)-pyridazinone | |
| SKF 96231 | 2-(2-propoxyphenyl)-6-purinone | 3, 4, 5 |
| SLX 2101 | | 5 |
| Sulmazole | U.S. Pat. No. 3,985,891 | 3 |
| T 0156 | 2-(2-methylpyridin-4-yl)methyl-4-(3,4,5-trimethoxyphenyl)-8-(pyrimidin- | 5 |
| | 2-yl)methoxy-1,2-dihydro-1-oxo-2,7-naphthyridine-3-carboxylic acid | |
| | methyl ester hydrochloride | |
| T 1032 | methyl-2-(4-aminophenyl)-1,2-dihydro-1-oxo-7-(2-pyridylmethoxy)-4- | 5 |
| | (3,4,5-trimethoxyphenyl)-3-isoquinoline carboxylate sulfate | |
| T 440 | 6,7-diethoxy-1-[1-(2-methoxyethyl)-2-oxo-1,2-dihydropyridin-4- | 4 |
| | yl]naphthalene-2,3-dimethanol | |
| Tadalafil | (6R,12aR)-6-(1,3-benzodioxol-5-yl)-2-methyl-2,3,6,7,12,12a- | 4, 5 |
| | hexahydropyrazino[1,2,1,6]pyrido[3,4-b]indole-1,4-dione | |
| Tetomilast | 6-(2-(3,4-diethoxyphenyl)-4-thiazolyl)-2-pyridinecarboxylic acid | 4 |
| Theophylline | 3,7-dihydro-1,3-dimethyl-1H-purine-2,6-dione | Not selective |
| Tibenelast | 5,6-diethoxybenzo(B)thiophene-2-carboxylic acid | 4 |
| Toborinone | (+/−)-6-[3-(3,4-dimethoxybenzylamino)-2-hydroxypropoxy]-2(1H)- | 3 |
| | quinolinone | |
| Tofimilast | 9-cyclopentyl-7-ethyl-6,9-dihydro-3-(2-thienyl)-5H-pyrazolo(3,4-c)-1,2,4- | 4 |
| | triazolo(4,3-a)pyridine | |
| Tolafentrine | N-[4-[(4aS,10bR)-8,9-dimethoxy-2-methyl-3,4,4a,10b-tetrahydro-1H- | 3, 4 |
| | pyrido[4,3-c]isoquinolin-6-yl]phenyl]-4-methylbenzenesulfonamide | |
| Torbafylline | 7-(ethoxymethyl)-3,7-dihydro-1-(5-hydroxy-5-methylhexyl)-3-methyl-1-H- | 4 |
| | purine-2,6-dione | |
| Trequinsin | 2,3,6,7-tetrahydro-9,10-dimethoxy-3-methyl-2-((2,4,6- | 2, 3, 4 |
| | trimethylphenyl)imino)-4H-pyrimido(6,1-a)isoquinolin-4-one | |
| UCB 29936 | | 4 |
| UDCG 212 | 5-methyl-6-[2-(4-oxo-1-cyclohexa-2,5-dienylidene)-1,3- | 3 |
| | dihydrobenzimidazol-5-yl]-4,5-dihydro-2H-pyridazin-3-one | |
| Udenafil | 3-(1-methyl-7-oxo-3-propyl-4H-pyrazolo[5,4-e]pyrimidin-5-yl)-N-[2-(1- | 5 |
| | methylpyrrolidin-2-yl)ethyl]-4-propoxybenzenesulfonamide | |
| UK 114542 | 5-[2-ethoxy-5-(morpholinylacetyl) phenyl]-1,6-dihydro-1-methyl-3-propyl- | 5 |
| | 7H-pyrazolo [4,3-d]-pyrimidin-7-one | |
| UK 343664 | 3-ethyl-5-(5-((4-ethylpiperazino)sulphonyl)-2-propoxyphenyl)-2-(2- | 5 |
| | pyridylmethyl)-6,7-dihydro-2H-pyrazolo(4,3-d)pyrimidin-7-one | |
| UK 357903 | 1-ethyl-4-{3-[3-ethyl-6,7-dihydro-7-oxo-2-(2-pyridylmethyl)-2H- | 5 |
| | pyrazolo[4,3-d] pyrimidin-5-yl]-2-(2-methoxyethoxy)5-pyridylsulphonyl}piperazine | |
| UK 369003 | | 5 |
| V 11294A | 3-((3-(cyclopentyloxy)-4-methoxyphenyl)methyl)-N-ethyl-8-(1- | 4 |
| | methylethyl)-3H-purin-6-amine monohydrochloride | |
| Vardenafil | 2-(2-ethoxy-5-(4-ethylpiperazin-1-yl-1-sulfonyl)phenyl)-5-methyl-7- | 5 |
| | propyl-3H-imidazo(5,1-f)(1,2,4)triazin-4-one | |
| Vesnarinone | U.S. Pat. No. 4,415,572 | 3, 5 |
| Vinpocetine | (3-alpha,16-alpha)-eburnamenine-14-carboxylic acid ethyl ester | 1, 3, 4 |
| WAY 122331 | 1-aza-10-(3-cyclopentyloxy-4-methoxyphenyl)-7,8-dimethyl-3- | 4 |
| | oxaspiro[4.5]dec-7-en-2-one | |
| WAY 127093B | [(3S)-3-(3-cyclopentyloxy-4-methoxyphenyl)-2-methyl-5- | 4 |
| | oxopyrazolidinyl]-N-(3-pyridylmethyl)carboxamide | |
| WIN 58237 | 1-cyclopentyl-3-methyl-6-(4-pyridinyl)pyrazolo (3,4-d)pyrimidin-4(5H)- | 5 |
| | one | |
| WIN 58993 | 5-methyl-6-pyridin-4-yl-3H-[1,3]thiazolo[5,4-e]pyridin-2-one | 3 |
| WIN 62005 | 5-methyl-6-pyridin-4-yl-1,3-dihydroimidazo[4,5-e]pyridin-2-one | 3 |
| WIN 62582 | 6-pyridin-4-yl-5-(trifluoromethyl)-1,3-dihydroimidazo[4,5-b]pyridin-2-one | 3 |
| WIN 63291 | 6-methyl-2-oxo-5-quinolin-6-yl-1H-pyridine-3-carbonitrile | 3 |
| WIN 65579 | 1-cyclopentyl-6-(3-ethoxy-4-pyridinyl)-3-ethyl-1,7-dihydro-4H- | 5 |
| | pyrazolo[3,-4-d]pyrimidin-4-one | |
| Y 20487 | 6-(3,6-dihydro-2-oxo-2H-1,3,4-thiadiazin-5-yl)-3,4-dihydro-2(1H)- | 3 |
| | quinolinone | |
| YM 58997 | 4-(3-bromophenyl)-1,7-diethylpyrido[2,3-d]pyrimidin-2(1H)-one | 4 |
| YM 976 | 4-(3-chlorophenyl)-1,7-diethylpyrido(2,3-d)pyrimidin-2(1H)-one | 4 |
| Z 15370A | | 4 |
| Zaprinast | 1,4-dihydro-5-(2-propoxyphenyl)-7H-1,2,3-triazolo[4,5-d]pyrimidine-7- | 5 |
| | one | |
| Zaprinast | 2-o-propoxyphenyl-8-azapurine-6-one | 1, 5 |
| Zardaverine | 6-(4-(difluoromethoxy)-3-methoxyphenyl)-3(2H)-Pyridazinone | 3, 4 |
| Zindotrine | 8-methyl-6-(1-piperidinyl)-1,2,4-triazolo(4,3-b)pyridazine | |

Examples of PDE4 inhibitors, e.g., ibudilast, include pyrrolidinones, such as the compounds disclosed in U.S. Pat. No. 5,665,754, US20040152754 and US20040023945; quinazolineones, such as the compounds disclosed in U.S. Pat. No. 6,747,035, U.S. Pat. No. 6,828,315, PCT publications WO 97/49702 and WO 97/42174; xanthine derivatives; phenylpyridines, such as the compounds disclosed in U.S. Pat. No. 6,410,547, U.S. Pat. No. 6,090,817, and PCT publication WO 97/22585; diazepine derivatives, such as the compounds disclosed in WO 97/36905; oxime derivatives, such as the compounds disclosed in U.S. Pat. No. 5,693,659 and PCT publication WO 96/00215; naphthyridines, such as the compounds described in U.S. Pat. No. 5,817,670, U.S. Pat. No. 6,740,662, U.S. Pat. No. U.S. Pat. No. 6,136,821, U.S. Pat. No. 6,331,548, U.S. Pat. No. 6,297,248, U.S. Pat. No. 6,541,480, U.S. Pat. No. 6,642,250, U.S. Pat. No. 6,900,205, Trifilieff et al. (Pharmacology 301: 241-248 (2002)) and Hersperger et al. (J. Med. Chem. 43:675-82(2000); benzofurans, such as the compounds disclosed in U.S. Pat. No. 5,902,824, U.S. Pat. No. 6,211,203, U.S. Pat. No. 6,514,996, U.S. Pat. No. 6,716,987, U.S. Pat. No. 6,376,535, U.S. Pat. No. 6,080,782, U.S. Pat. No. 6,054,475, EP 819688, EP 685479, and Perrier et al. (Bioorg. Med. Chem. Lett. 9:323-326 (1999)); phenanthridines, such as those disclosed in U.S. Pat. No. 6,191,138, U.S. Pat. No. 6,121,279, and U.S. Pat. No. 6,127,378; benzoxazoles, such as those disclosed in U.S. Pat. No. 6,166,041 and U.S. Pat. No. 6,376,485; purine derivatives, such as the compounds disclosed in U.S. Pat. No. 6,228,859; benzamides, such as the compounds described in U.S. Pat. No. 5,981,527, U.S. Pat. No. 5,712,298, PCT publications WO95/01338 and WO 97/48697, and Ashton et al. (J. Med. Chem. 37: 1696-1703 (1994)); substituted phenyl compounds, such as the compounds disclosed in U.S. Pat. No. 6,297,264, U.S. Pat. No. 5,866,593, U.S. Pat. No. 655,859,034, U.S. Pat. No. 6,245,774, U.S. Pat. No. 6,197,792, U.S. Pat. No. 6,080,790, U.S. Pat. No. 6,077,854, U.S. Pat. No. 5,962,483, U.S. Pat. No. 5,674,880, U.S. Pat. No. 5,786,354, U.S. Pat. No. 5,739,144, U.S. Pat. No. 5,776,958, U.S. Pat. No. 5,798,373, U.S. Pat. No. 5,891,896, U.S. Pat. No. 5,849,770, U.S. Pat. No. 5,550,137, U.S. Pat. No. 5,340,827, U.S. Pat. No. 5,780,478, U.S. Pat. No. 5,780,477, U.S. Pat. No. 5,633,257, and PCT publication WO 95/35283; substituted biphenyl compounds, such as those disclosed in U.S. Pat. No. 5,877,190; and quinilinones, such as the compounds described in U.S. Pat. No. 6,800,625 and PCT publication WO 98/14432.

Yet other inhibitors of PDE4 are disclosed in U.S. Pat. No. 6,716,987, U.S. Pat. No. 6,514,996, U.S. Pat. No. 6,740,655, U.S. Pat. No. 6,559,168, U.S. Pat. No. 6,069,151, U.S. Pat. No. 6,365,585, U.S. Pat. No. 6,313,116, U.S. Pat. No. 6,245,774, U.S. Pat. No. 6,011,037, U.S. Pat. No. 6,127,363, U.S. Pat. No. 6,303,789, U.S. Pat. No. 6,316,472, U.S. Pat. No. 6,348,602, U.S. Pat. No. 6,331,543, U.S. Pat. No. 6,333,354, U.S. Pat. No. 5,491,147, U.S. Pat. No. 5,608,070, U.S. Pat. No. 5,622,977, U.S. Pat. No. 5,580,888, U.S. Pat. No. 6,680,336, U.S. Pat. No. 6,569,890, U.S. Pat. No. 6,569,885, U.S. Pat. No. 6,500,856, U.S. Pat. No. 6,486,186, U.S. Pat. No. 6,458,787, U.S. Pat. No. 6,455,562, U.S. Pat. No. 6,444,671, U.S. Patent. No. 6,423,710, U.S. Pat. No. 6,376,489, U.S. Pat. No. 6,372,777, U.S. Pat. No. 6,362,213, U.S. Pat. No. 6,313,156, U.S. Pat. No. 6,294,561, U.S. Pat. No. 6,258,843, U.S. Pat. No. 6,258,833, U.S. Pat. No. 6,121,279, U.S. Pat. No. 6,043,263, U.S. Pat. No. 6,297,257, U.S. Pat. No. 6,251,923, U.S. Pat. No. 6,613,794, U.S. Pat. No. 6,407,108, U.S. Pat. No. 6,107,295, U.S. Pat. No. 6,103,718, U.S. Pat. No. 6,479,494, U.S. Pat. No. 6,602,890, U.S. Pat. No. 6,545,158, U.S. Pat. No. 6,545,025, U.S. Pat. No. 6,498,160, U.S. Pat. No. 6,743,802, U.S. Pat. No. 6,787,554, U.S. Pat. No. 6,828,333, U.S. Pat. No. 6,869,945, U.S. Pat. No. 6,894,041, U.S. Pat. No. 6,924,292, U.S. Pat. No. 6,949,573, U.S. Pat. No. 6,953,810, U.S. Pat. No. 5,972,927, U.S. Pat. No. 5,962,492, U.S. Pat. No. 5,814,651, U.S. Pat. No. 5,723,460, U.S. Pat. No. 5,716,967, U.S. Pat. No. 5,686,434, U.S. Pat. No. 5,502,072, U.S. Pat. No. 5,116,837, U.S. Pat. No. 5,091,431; U.S. Pat. No. 4,670,434; U.S. Pat. No. 4,490,371, U.S. Pat. No. 5,710,160, U.S. Pat. No. 5,710,170, U.S. Pat. No. 6,384,236; in U.S. Patent publications 2005/0119225 and 2005/0026913; in PCT publications WO 99/65880, WO 00/26201, WO 98/06704, WO 00/59890, WO9907704, WO9422852, WO 98/20007, WO 02/096423, WO 98/18796, WO 98/02440, WO 02/096463, WO 97/44337, WO 97/44036, and WO 97/44322; in European patent EP 0763534; and in Aoki et al. (J. Pharmacol. Exp. Ther. 295:255-60 (2000)), Del Piaz et al. (Eur. J. Med. Chem. 35:463-480 (2000)), and Barnette et al. (Pharmacol. Rev. Commun. 8:65-73 (1997)).

Certain PDE inhibitors are described in more detail below.

Etazolate

The compound 1-ethyl-4-((1-methylethylidene)hydrazino)-1H-pyrazolo(3,4-b)pyridine-5-carboxylic acidethyl ester, also known as etazolate, is a PDE inhibitor having the structure:

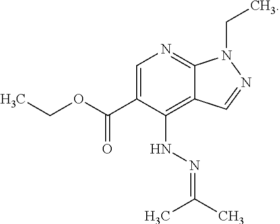

Exemplary analogs of etazolate are ethyl 1-methyl-4-(2-propan-2-ylidenehydrazinyl)pyrazolo[3,4-b]pyridine-5-carboxylate, butyl 1-ethyl-4-(2-propan-2-ylidenehydrazinyl)pyrazolo[3,4-b]pyridine-5-carboxylate, ethyl 1-propan-2-yl-4-(2-propan-2-ylidenehydrazinyl)pyrazolo[3,4-b]pyridine-5-carboxylate, ethyl 4-(2-cyclohexylidenehydrazinyl)-1-ethylpyrazolo[3,4-b]pyridine-5-carboxylate, ethyl 1-ethyl-4-(2-nonan-5-ylidenehydrazinyl)pyrazolo[3,4-b]pyridine-5-carboxylate, butyl 1-ethyl-4-hydrazinylpyrazolo[3,4-b]pyridine-5-carboxylate, ethyl 4-hydrazinyl-1-propan-2-ylpyrazolo[3,4-b]pyridine-5-carboxylate, ethyl 1-ethyl-4-hydrazinylpyrazolo[3,4-b]pyridine-5-carboxylate, ethyl 4-hydrazinyl-1-methylpyrazolo[3,4-b]pyridine-5-carboxylate, ethyl 4-amino-1-methylpyrazolo[3,4-b]pyridine-5-carboxylate, ethyl 4-[(2E)-2-(4,4-dimethoxybutan-2-ylidene)hydrazinyl]-1-ethylpyrazolo[3,4-b]pyridine-5-carboxylate, ethyl 4-[2-(1,3-dihydroxypropan-2-ylidene)hydrazinyl]-1-ethylpyrazolo[3,4-b]pyridine-5-carboxylate, and ethyl 4-(butylamino)-1-methylpyrazolo[3,4-b]pyridine-5-carboxylate.

Papaverine

The PDE inhibitor papaverine has the following structure:

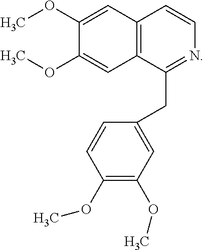

Analogs of papaverine include 3-(3,4-dimethoxyphenyl)-6,7-dimethoxyisoquinoline, 1-[1-(3,4-dimethoxyphenyl)ethenyl]-6,7-dimethoxyisoquinoline, 1-(3,4-dimethoxyphenyl)-6,7-dimethoxyisoquinoline, 1-[1-(3,4-dimethoxyphenyl)ethyl]-6,7-dimethoxyisoquinoline, 1-[1-(3,4-dimethoxyphenyl)ethyl]-6,7-dimethoxyisoquinoline, 6,7-dimethoxy-1-[(4-methoxyphenyl)methyl]isoquinoline, 6,7-dimethoxy-1-[(3-methoxyphenyl)methyl]isoquinoline, 6,7-dimethoxy-3-(4-methoxyphenyl)isoquinoline, 1-[(2,3-dimethoxyphenyl)methyl]-6,7-dimethoxyisoquinoline, 1-[(3,4-dimethoxyphenyl)methyl]-6,7-dimethoxy-2-methylisoquinolin-2-ium, and 1-[(3,4-dimethoxyphenyl)methyl]-5,6-dimethoxyisoquinoline. Other papaverine analogs are described in Shepard and Noth (J. Org. Chem. 19:415-418 (1954)).

Ethaverine

Ethaverine is the tetraethoxy analogue of papaverine and is described in U.S. Pat. No. 1,962,224. Ethaverine has the structure:

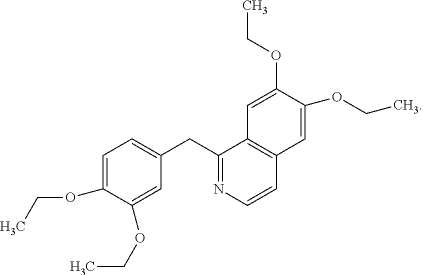

Analogs of ethaverine include without limitation 1-(3,4-diethoxyphenyl)-6,7-diethoxyisoquinoline, 6,7-dimethoxy-1-[(3-methoxy-4-propoxyphenyl)methyl]isoquinoline, 1-[(4-ethoxy-3-methoxyphenyl)methyl]-6,7-dimethoxyisoquinoline, 6,7-dimethoxy-1-[(3-methoxy-4-propoxyphenyl)methyl]isoquinoline hydrochloride, 1-[(3,4-diethoxyphenyl)methyl]-6,7-diethoxyisoquinoline hydrochloride, 1-[(3,4-diethoxyphenyl)methyl]-6,7-di(propan-2-yloxy)isoquinoline, 1-[(2,3-dimethoxyphenyl)methyl]-5,6-diethoxyisoquinoline, 1-[(3,4-diethoxyphenyl)methyl]-6,7-diethoxy-2-methylisoquinolin-2-ium, 1-[(2,3-dimethoxyphenyl)methyl]-5,6-diethoxyisoquinoline hydrochloride, and 1-(2,3-dimethoxyphenyl)-5,6-diethoxyisoquinoline.

EHNA

EHNA (9-(2-hydroxy-3-nonyl)adenine) is a PDE2-selective inhibitor having the following structure:

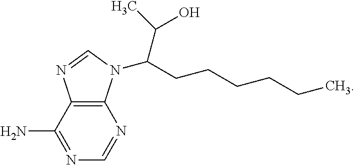

Exemplary analogs of EHNA are described by formula I of U.S. Pat. No. 7,022,709 and by formula I of U.S. Pat. No. 5,861,396. Other analogs of EHNA include 1,3-dideaza-EHNA, 7-deaza-EHNA, 1-deaza-EHNA, 3-deaza-EHNA, and erythro-(3-nonyl-p-aminobenzyl-adenine).

Drotaverine

Drotaverine (1-benzyl-3′,4′,6,7-tetraethoxy-1,2,3,4-tetrahydroisoquinoline) is a PDE4-selective PDE inhibitor structurally related to papaverine. Drotaverine is described in Belgium Patent No. 621,917 and has the following structure:

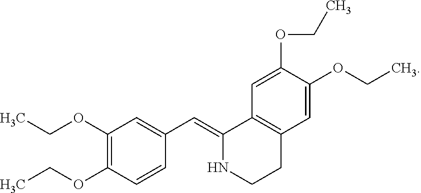

Trequinsin

Trequinsin (9,10-dimethoxy-3-methyl-2-(2,4,6-trimethylphenyl)imino-6,7-dihydropyrimido[6,1-a]isoquinolin-4-one) is an inhibitor of PDE3. The structure of trequinsin is:

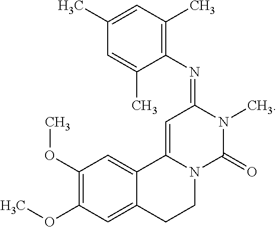

Analogs of trequinsin include 3-ethyl-9,10-dimethoxy-2-(2,4,6-trimethylphenyl)imino-6,7-dihydropyrimido[6,1-a]isoquinolin-4-one, (7)-9,10-dimethoxy-3,7-dimethyl-2-(2,4,6-trimethylphenyl)imino-6,7-dihydropyrimido[6,1-a]isoquinolin-4-one, 9,10-dimethoxy-3,7-dimethyl-2-(2,4,6-trimethylphenyl)imino-6,7-dihydropyrimido[6,1-a]isoquinolin-4-one, 9,10-dimethoxy-3-propan-2-yl-2-(2,4,6-trimethylphenyl)imino-6,7-dihydropyrimido[6,1-a]isoquinolin-4-one, (6)-6-ethyl-9,10-dimethoxy-3-methyl-2-(2,4,6-trimethylphenyl)imino-6,7-dihydropyrimido[6,1-a]isoquinolin-4-one, 9,10-dimethoxy-3,7,7-trimethyl-2-(2,4,6-trimethylphenyl)imino-6H-pyrimido[6,1-a]isoquinolin-4-one, 6-ethyl-9,10-dimethoxy-3-methyl-2-(2,4,6-trimethylphenyl)imino-6,7-dihydropyrimido[6,1-a]isoquinolin-4-one, 9,10-dimethoxy-3,6,7-trimethyl-2-(2,4,6-trimethylphenyl)imino-6,7-dihydropyrimido[6,1-a]isoquinolin-4-one, 9,10-dimethoxy-7-methyl-3-propyl-2-(2,4,6-trimethylphenyl)imino-6,7-dihydropyrimido[6,1-a]isoquinolin-4-one, 2-(2,4-dimethylphenyl)imino-9,10-dimethoxy-3,7-dimethyl-6,7-dihydropyrimido[6,1-a]isoquinolin-4-one, 7,7-diethyl-9,10-dimethoxy-3-methyl-2-(2,4,6-trimethylphenyl)imino-6H-pyrimido[6,1-a]isoquinolin-4-one, (6,7)-9,10-dimethoxy-3,6,7-trimethyl-2-(2,4,6-trimethylphenyl)imino-6,7-dihydropyrimido[6,1-a]isoquinolin-4-one, 9,10-dimethoxy-1,3-dimethyl-2-(2,4,6-trimethylphenyl)imino-6,7-dihydropyrimido[6,1-a]isoquinolin-4-one, 7,7-diethyl-9,10-dimethoxy-3-methyl-2-(2,4,6-trimethylphenyl)imino-6H-pyrimido[6,1-a]isoquinolin-4-one hydrochloride, 9,10-dimethoxy-3,7,7-trimethyl-2-(2,4,6-trimethylphenyl)imino-6H-pyrimido[6,1-a]isoquinolin-4-one hydrochloride, 2-(2,6-dimethylphenyl)imino-9,10-dimethoxy-3,7-dimethyl-6,7-dihydropyrimido[6,1-a]isoquinolin-4-one, and 2-(2,6-diethylphenyl)imino-9,10-dimethoxy-3,7-dimethyl-6,7-dihydropyrimido[6,1-a]isoquinolin-4-one.

The structures of additional PDE inhibitors that may be particularly useful are shown below.

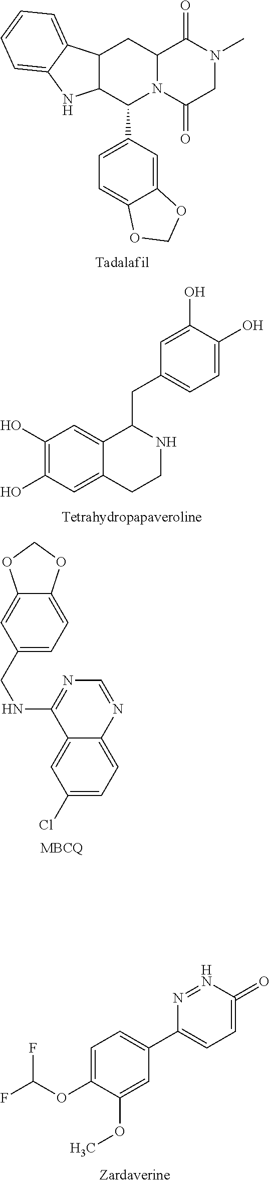

### CoQ10 Analogs

In certain embodiments, the methods, compositions, and kits of the invention employ idebenone, a CoQ10 (ubiquinone) analog. Analogs of idebenone include other CoQ10 analogs, e.g., MitoQ10, decyl-ubiquinone and atovaquone.

Idebenone

Idebenone is described in German Patent No. 2,130,794 and U.S. Pat. No. 4,271,083 and has the structure:

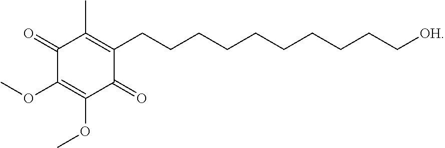

Analogs of idebenone are described by formulae I-IV of U.S. Pat. No. 4,271,083, e.g., 2,3,5-trimethyl-6-(6′-hydroxyhexyl)-1,4-benzoquinone, 2,3-dimethoxy-5-methyl-6-(4′-hydroxybutyl)-1,4-benzoquinone, 2,3,5-trimethyl-6-(6′-hydroxy-1′-oxohexyl)-1,4-benzoquinone, 2,3,5-trimethyl-6-(1′,6′-dihydroxyhexyl)-1,4-benzoquinone, 2,3,5-trimethyl-6-(6′-hydroxyhexyl)-1,4-benzoquinone, 2,3-dimethoxy-5-methyl-6-(10′-hydroxydecyl)-1,4-benzoquinone, and 2,3-dimethoxy-5-methyl-6-(10′-hydroxydecyl)-1,4-benzoquinone; by formula I of U.S. Pat. No. 4,484,000, e.g., 2′,5′-bis-(5-methoxycarbonyl-2-methylpent-2-yl)-hydroquinone and 2′,5-bis-(5-carboxy-2-methyl-pent-2-yl)hydroquinone, di(n-hexyl)ester; by formula I of U.S. Pat. No. 4,514,420, e.g., 2,3-dimethoxy-5-methyl-6-(10′-hydroxydecyl)-1,4-benzoquinone; by formula I of U.S. Pat. No. 4,526,719, e.g., 4-[4-(6-(2,3-dimethoxy-5-methyl-1,4-benzoquinonyl))-2-methyl-2-butenoxy]cinnamic acid, 3-[6-(2,3-dimethoxy-5-methyl-1,4-benzoquinonyl)]-acrylic acid, and 1-[6-(2,3-dimethoxy-5-methyl]-1,4-benzoquinonyl)]3-oxo-1-butene; by formula I of U.S. Pat. No. 4,985,447, e.g., 3,5,6-trimethyl-2-(3-pyridyl-2-thienylmethyl)-1,4-benzoquinone hydrochloride and 7-(3,5,6-trimethyl-1,4-benzoquinon-2-yl)-7-(3-pyridyl)pheptanoic acid; by formula I of U.S. Pat. No. 5,106,858, e.g., 3,5,6-trimethyl-2-(3-pyridyl)methyl-1,4-benzoquinone hydrochloride, 3,5,6-trimethyl-2-[1-(3-pyridyl)ethyl]-1,4-benzoquinone, 7-(3,5,6-trimethyl-1,4-benzoquinon-2-yl)-7-phenylheptanoic acid, 6-(3,5,6-trimethyl-1,4-benzoquinon-2-yl)-6-(4-methoxyphenyl)hexanoic acid, 7-(3,5,6-trimethyl-1,4-benzoquinon-2-yl)-7-(4-methoxyphenyl)heptanoic acid, 7-(3,5,6-trimethyl-1,4-benzoquinon-2-yl)-7-(4-fluorophenyl)heptanoic acid, 7-(3,5,6-trimetyl-1,4-benzoquinon-2-yl)-7-(4-methylphenyl)heptanoic acid, and 2-[(1-imidazolyl)methyl]-3,5,6-trimethyl-1,4-benzoquinone hydrochloride; and by U.S. Pat. No. 5,304,658, e.g., 7-(3,5,6-trimethyl-1,4-benzoquinon-2-yl)-7-phenylheptanol, 7-(3,5,6-trimethyl-1,4-benzoquinon-2-yl)-7-phenylheptanamide, 7-(3,5,6-trimethyl-1,4-benzoquinon-2-yl)-7-phenylheptanoglycine, and 1-[7-(3,5,6-trimethyl-1,4-benzoquinon-2-yl)-7-phenylheptanoyl]4-(2-phenylethyl)piperadine.

### Calcium Channel Blockers

Verapamil may be used in the methods, compositions, and kits of the invention. Analogs of verapamil include structural analogs of verapamil and other calcium channel blockers, e.g., dihydropyridines (e.g., amlodipine, aranidipine, azelnidipine, barnidipine, benidipine, cilnidipine, clevidipine, efonidipine, felodipine, lacidipine, lercanidipine, manidipine, nicardipine, nifedipine, nilvadipine, nimodipine, nisoldipine, nitrendipine, and pranidipine), phenylalkylamines (e.g., gallopamil), and benzothiazepines (e.g., diltiazem). Other examples are dilazep (described above), bepridil, lomerizine, mibefradil, fluspirilene, and fendiline.

Verapamil

Verapamil is described in Belgian Patent No. 615,861 and in U.S. Pat. No. 3,261,859 and has the structure:

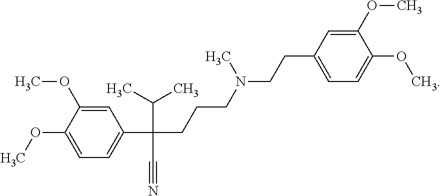

Analogs of verapamil include 4-desmethoxy-verapamil, 2-(3,4-dimethoxyphenyl)-5-amino-2-isopropylvaleronitrile, alpha-(3-aminopropyl)-3,4-dimethoxy-alpha-(1-methylethyl)benzeneacetonitrile, carboxyverapamil, devapamil, norgallopamil, and nexopamil.

### Bisphosphonates

A bisphosonate, also called a diphosphonate, may be employed in the methods, compositions, and kits of the invention. Bisphosphonates are a class of drugs that inhibits bone resporption. Examples of bisphonates are described below.

Pamindronate and Alendronate

Pamidronic acid is described in German Patent No. 2,130,794 and U.S. Pat. No. 4,327,039, and alendronic acid is described in Belgian Patent No. 903,519 and U.S. Pat. No. 4,705,651. The structures of alendronate and pamidronate are:

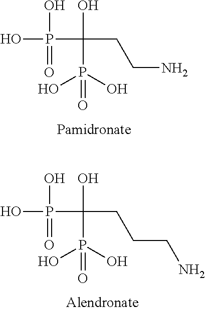

Exemplary analogs of alendronate and pamidronate are etidronate, clodronate, tiludronate, risedronate, ibandronate, EB-1053 (1-hydroxy-3-(1-pyrrolidinyl)-propylidene-1,1-bisphosphonate), olpadronate, amino-olpadronate, 6-amino-1-hydroxyhexylidene-bisphosphonate, cimadronate, neridronate, piridronate, zoledronate, and 1-hydroxy-3(methylpentylamino)-propylidene bisphosphonate. Other exemplary analogs are described by the general formula of U.S. Pat. No. 4,327,039; by formula I of U.S. Pat. No. 4,407,761, e.g., 6-amino-1-hydroxyhexylidene-1,1-bisphosphonic acid; by formula I of U.S. Pat. No. 4,536,348, e.g., 1,3-dihydroxypropane-1,1-diphosphonic acid and 1,6-dihydroxyhexane-1,1-diphosphonic acid; by formula I of U.S. Pat. No. 5,227,506, e.g., 4-amino-1-hydroxybutylidene-1,1-bisphosphonic acid mono(pivaloyloxymethyl)ester, 4-amino-1-hydroxybutylidene-1,1-bisphosphonic acid di(pivaloyloxymethyl)ester, 4-amino-1-hydroxybutylidene-1,1-bisphosphonic acid tri(pivaloyloxymethyl)ester, 4-amino-1-hydroxybutylidene-1,1-bisphosphonic acid tetra(pivaloyloxymethyl)ester, 4-amino-1-hydroxybutylidene-1,1-bisphosphonic acid di(pivaloyloxymethyl)ester monosodium salt, 4-amino-1-hydroxybutylidene-1,1-bisphosphonic acid di[(2-ethyl)butanoyloxymethyl]ester, 4-amino-1-hydroxybutylidene-1,1-bisphosphonic acid tri[(2-ethyl)butanoyloxymethyl]ester, 4-amino-1-hydroxybutylidene-1,1-bisphosphonic acid tri(2,2-dimethylbutanoyloxymethyl)ester, and 4-amino-1-hydroxybutylidene-1,1-bisphosphonic acid tri(isobutanoyloxymethyl)ester, N-methyl-4-amino-1-hydroxybutylidene-1,1-bisphosphonic acid di(pivaloyloxymethyl)ester, N-methyl-4-amino-1-hydroxybutylidene-1,1-bisphosphonic acid tri(pivaloyloxymethyl)ester, N-methyl-4-amino-1-hydroxybutylidene-1,1-bisphosphonic acid di[(2-ethyl)butanoyloxymethyl], ester, N-methyl-4-amino-1-hydroxybutylidene-1,1-bisphosphonic acid tri[(2-ethyl)butanoyloxymethyl]ester, 4-(N,N-dimethylamino)-1-hydroxybutylidene-1,1-bisphosphonic acid tri(pivaloyloxymethyl)ester, 3-amino-1-hydroxypropylidene-1,1-bisphosphonic acid tri(pivaloyloxymethyl)ester, 3-(N,N-dimethylamino)-1-hydroxypropylidene-1,1-bisphosphonic acid tri(pivaloyloxymethyl)ester, 1-hydroxy-3-(N-methyl-N-pentylamino)propylidene-1,1-bisphosphonic acid tri(pivaloyloxymethyl)ester, 1-hydroxy-2-[3-pyridyl]ethylidene-bisphosphonic acid tri(pivaloyloxymethyl)ester, 4-(hydroxymethylene-bisphosphonic acid)piperidine tri(pivaloyloxymethyl)ester, 1-hydroxyethylidene-1,1-bisphosphonic acid tri(pivaloyloxymethyl)ester, 1-hydroxyethylidene-1,1-bisphosphonic acid tetra(pivaloyloxymethyl)ester, [(4-chlorophenyl)thio]methylene-bisphosphonic acid tri(pivaloyloxymethyl)ester, [(4-chlorophenyl)thio]methylene-bisphosphonic acid tetra(pivaloyloxymethyl)ester, dichloromethylene-bisphosphonic acid tetra(pivaloyloxymethyl)ester, difluoromethylene-bisphosphonic acid tetra(pivaloyloxymethyl)ester, and methylene-bisphosphonic acid tetra(pivaloyloxymethyl)ester; by the general formula of U.S. Pat. No. 5,583,122, e.g., risedronate, 2-(2-pyridyl)-ethane-1,1-diphosphonic acid, 2-(3-pyridyl)-ethane-1,1-diphosphonic acid, 2-(4-pyridyl)-ethane-1,1-diphosphonic acid, 2-(2-pyridyl)-hydroxyethane-1,1-diphosphonic acid, 2-(3-pyridyl)-hydroxyethane-1,1-diphosphonic acid, and 2-(4-pyridyl)-hydroxyethane-1,1-diphosphonic acid; by formula I of U.S. Pat. No. 4,927,814, e.g., 1-hydroxy-3-(N-methyl-N-nonylamino)-propane-1,1-diphosphonic acid, 1-hydroxy-3-(N-methyl-N-pentylamino)-propane-1,1-diphosphonic acid, 1-hydroxy-3-(N-isobutyl-N-methylamino)-propane-1,1-diphosphonic acid; by formula I of U.S. Pat. No. 4939130, e.g., 2-(imidazol-1-yl)-1-hydroxy-ethane-1,1-diphosphonic acid and 2-(1-methylimidazol-2-yl)-1-hydroxyethane-1,1-diphosphonic acid; by formula I of U.S. Pat. No. 4,876,248, e.g., tetramethyl benzoxazol-2-yl-thiomethylene-diphosphonate (SR 41625), tetraisopropyl 4-phenylthio-butylene-1,1-diphosphonate (SR 41341), tetraisopropyl n-octylthiomethylene-diphosphonate (SR 41454), tetraisopropyl 7-(4-nitrophenylthio)-heptylidene-1,1-diphosphonate (SR 42147), tetraisopropyl (3-phenyl-propylthio)-methylene-diphosphonate (SR 41907), tetraethyl (N,N-diethylthiocarbamylthio)-methylene-diphosphonate (SR 41905), tetraisopropyl perfluorohexylthio-methylene-disphosphonate (SR 42327), tri-(tertiary butylamine) salt of methylthio-methylene-diphosphonic acid (SR 41036), di-(tertiary butylamine) salt of (4-chlorophenyl)thiomethylene-diphosphonic acid (SR 41319), tertiary butylamine salt of 3-methylthio-propylidene-1,1-diphosphonic acid (SR 41273), di-(tertiary butylamine salt) of 4-phenylthio-butylidene-1,1-diphosphonic acid (SR 41342), monoammonium hexadecyithiomethylene-diphosphonate (SR 41453), di-(tertiary butylamine) salt of (2-hydroxyethylthio)methylene-diphosphonic acid (SR 41318), disodium methylthiomethylene-diphosphonate (SR 41553), tri-(tertiary butylamine) salt of benzothiazol-2-yl-thiomethylene-diphosphonic acid (SR 41481), tertiary-butylammonium 4-(methylthio)-butylidene-1,1-diphosphonate (SR 41177), di-(tertiary butylalmine) salt of 5-mercapto-pentylidene-1,1-diphosphonic acid (SR 41527), di-(tertiary butylamine) salt of 7-(1-methyl-imidazol-2-yl-thio)-heptylidene-1,1-diphosphonic acid (SR 42132), tetraethyl 5-(4-fluoro-phenylthio)-1-hydroxy-pentylidene-1,1-diphosphonate (SR 41906), tetraethyl 5-(pyrid-2-yl-thio)-1-hydroxy-pentylidene-1,1-diphosphonate (SR 42090), and di-(tertiary butylamine) salt of 5-(4-fluorophenylthio)-1-hydroxy-pentylidene-1,1-diphosphonic acid (SR 41909); and in U.S. Pat. No. 3,159,581.

### Conjugates

If desired, the agents used in any of the combinations described herein may be covalently attached to one another to form a conjugate of formula I.

(A)-(L)-(B) (I)

In formula I, (A) is a Compound A and (B) is Compound B of a pair of agents from e.g., Table 1, and L is a covalent linker that tethers (A) to (B). Conjugates of the invention can be administered to a subject by any route and for the treatment of muscular dystrophy.

The conjugates of the invention can be prodrugs, releasing drug (A) and drug (B) upon, for example, cleavage of the conjugate by intracellular and extracellular enzymes (e.g., amidases, esterases, and phosphatases). The conjugates of the invention can also be designed to largely remain intact in vivo, resisting cleavage by intracellular and extracellular enzymes. The degradation of the conjugate in vivo can be controlled by the design of linker (L) and the covalent bonds formed with drug (A) and drug (B) during the synthesis of the conjugate.

Conjugates can be prepared using techniques familiar to those skilled in the art. For example, the conjugates can be prepared using the methods disclosed in G. Hermanson, Bioconjugate Techniques, Academic Press, Inc., 1996. The synthesis of conjugates may involve the selective protection and deprotection of alcohols, amines, ketones, sulfhydryls or carboxyl functional groups of drug (A), the linker, and/or drug (B). For example, commonly used protecting groups for amines include carbamates, such as tent-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a lewis acid, or hydrogenation) are required to remove each, exclusive of other protecting groups in a molecule. The conditions required for the addition of protecting groups to amine, alcohol, sulfhydryl, and carboxyl functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994. Additional synthetic details are provided below.

### Linkers

The linker component of the invention is, at its simplest, a bond between drug (A) and drug (B), but typically provides a linear, cyclic, or branched molecular skeleton having pendant groups covalently linking drug (A) to drug (B).

Thus, linking of drug (A) to drug (B) is achieved by covalent means, involving bond formation with one or more functional groups located on drug (A) and drug (B). Examples of chemically reactive functional groups which may be employed for this purpose include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

The covalent linking of drug (A) and drug (B) may be effected using a linker that contains reactive moieties capable of reaction with such functional groups present in drug (A) and drug (B). For example, an amine group of drug (A) may react with a carboxyl group of the linker, or an activated derivative thereof, resulting in the formation of an amide linking the two.

Examples of moieties capable of reaction with sulfhydryl groups include α-haloacetyl compounds of the type XCH₂CO— (where X═Br, Cl, or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but may additionally be useful in coupling to amino groups under certain conditions. Reagents such as 2-iminothiolane (Traut et al., Biochemistry 12:3266 (1973)), which introduce a thiol group through conversion of an amino group, may be considered as sulfhydryl reagents if linking occurs through the formation of disulfide bridges.

Examples of reactive moieties capable of reaction with amino groups include, for example, alkylating and acylating agents. Representative alkylating agents include:

(i) α-haloacetyl compounds, which show specificity towards amino groups in the absence of reactive thiol groups and are of the type XCH₂CO— (where X═Br, Cl, or I), for example, as described by Wong Biochemistry 24:5337 (1979);

(ii) N-maleimide derivatives, which may react with amino groups either through a Michael type reaction or through acylation by addition to the ring carbonyl group, for example, as described by Smyth et al., J. Am. Chem. Soc. 82:4600 (1960) and Biochem. J. 91:589 (1964);

(iii) aryl halides such as reactive nitrohaloaromatic compounds;

(iv) alkyl halides, as described, for example, by McKenzie et al., J. Protein Chem. 7:581 (1988);

(v) aldehydes and ketones capable of Schiff's base formation with amino groups, the adducts formed usually being stabilized through reduction to give a stable amine;

(vi) epoxide derivatives such as epichlorohydrin and bisoxiranes, which may react with amino, sulfhydryl, or phenolic hydroxyl groups;

(vii) chlorine-containing derivatives of s-triazines, which are very reactive towards nucleophiles such as amino, sufhydryl, and hydroxyl groups;

(viii) aziridines based on s-triazine compounds detailed above, e.g., as described by Ross, J. Adv. Cancer Res. 2:1 (1954), which react with nucleophiles such as amino groups by ring opening;

(ix) squaric acid diethyl esters as described by Tietze, Chem. Ber. 124:1215 (1991); and

(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include:

(i) isocyanates and isothiocyanates, particularly aromatic derivatives, which form stable urea and thiourea derivatives respectively;

(ii) sulfonyl chlorides, which have been described by Herzig et al., Biopolymers 2:349 (1964);

(iii) acid halides;

(iv) active esters such as nitrophenylesters or N-hydroxysuccinimidyl esters;

(v) acid anhydrides such as mixed, symmetrical, or N-carboxyanhydrides;

(vi) other useful reagents for amide bond formation, for example, as described by M. Bodansky, Principles of Peptide Synthesis, Springer-Verlag, 1984;

(vii) acylazides, e.g., wherein the azide group is generated from a preformed hydrazide derivative using sodium nitrite, as described by Wetz et al., Anal. Biochem. 58:347 (1974); and

(viii) imidoesters, which form stable amidines on reaction with amino groups, for example, as described by Hunter and Ludwig, J. Am. Chem. Soc. 84:3491 (1962).

Aldehydes and ketones may be reacted with amines to form Schiff's bases, which may advantageously be stabilized through reductive amination. Alkoxylamino moieties readily react with ketones and aldehydes to produce stable alkoxamines, for example, as described by Webb et al., in Bioconjugate Chem. 1:96 (1990).

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through O-acylurea formation followed by amide bond formation, may also be employed.

It will be appreciated that functional groups in drug (A) and/or drug (B) may, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

So-called zero-length linkers, involving direct covalent joining of a reactive chemical group of drug (A) with a reactive chemical group of drug (B) without introducing additional linking material may, if desired, be used in accordance with the invention.

More commonly, however, the linker will include two or more reactive moieties, as described above, connected by a spacer element. The presence of such a spacer permits bifunctional linkers to react with specific functional groups within drug (A) and drug (B), resulting in a covalent linkage between the two. The reactive moieties in a linker may be the same (homobifunctional linker) or different (heterobifunctional linker, or, where several dissimilar reactive moieties are present, heteromultifunctional linker), providing a diversity of potential reagents that may bring about covalent attachment between drug (A) and drug (B).

Spacer elements in the linker typically consist of linear or branched chains and may include a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl.

In some instances, the linker is described by formula (V):

G¹-(Z¹)ₒ—(Y¹)ᵤ—(Z²)ₛ—(R₃₀)—(Z³)ₜ—(Y²)ᵥ—(Z⁴)ₚ-G² (V)

In formula (V), G¹ is a bond between drug (A) and the linker; G² is a bond between the linker and drug (B); Z¹, Z², Z³, and Z⁴ each, independently, is selected from O, S, and NR₃₁; R₃₁ is hydrogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; Y¹ and Y² are each, independently, selected from carbonyl, thiocarbonyl, sulphonyl, or phosphoryl; o, p, s, t, u, and v are each, independently, 0 or 1; and R₃₀ is a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl, or a chemical bond linking G¹-(Z¹)ₒ—(Y¹)ᵤ—(Z²)ₛ— to —(Z³)ₜ—(Y²)ᵥ—(Z⁴)ₚ-G².

Examples of homobifunctional linkers useful in the preparation of conjugates of the invention include, without limitation, diamines and diols selected from ethylenediamine, propylenediamine and hexamethylenediamine, ethylene glycol, diethylene glycol, propylene glycol, 1,4-butanediol, 1,6-hexanediol, cyclohexanediol, and polycaprolactone diol.

### Formulation of Pharmaceutical Compositions

The compositions, methods, and kits of the invention can include formulation(s) of compound(s) that, upon administration to a subject, result in a concentration of the compound(s) that treats muscular dystrophy. The compound(s) may be contained in any appropriate amount in any suitable carrier substance, and are generally present in an amount of 1-95% by weight of the total weight of the composition. The composition may be provided in a dosage form that is suitable for the oral, parenteral (e.g., intravenously or intramuscularly), rectal, dermatological, cutaneous, nasal, vaginal, inhalant, skin (patch), ocular, intrathecal, or intracranial administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions according to the invention or used in the methods of the invention may be formulated to release the active compound immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agent(s) of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the combination to a particular target cell type. Administration of compound(s) in the form of a controlled release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound(s) are formulated with appropriate excipients into a pharmaceutical composition that, upon administration, releases the compound(s) in a controlled manner. Examples include single or multiple unit tablet or capsule compositions, oil solutions, suspensions, emulsions, microcapsules, molecular complexes, microspheres, nanoparticles, patches, and liposomes.

### Delivery of Compound(s)

It is not intended that administration of compounds be limited to a single formulation and delivery method for all compounds of a combination. The combination can be administered using separate formulations and/or delivery methods for each compound of the combination using, for example, any of the above-described formulations and methods. In one example, a first agent is delivered orally, and a second agent is delivered intravenously.

### Dosages

The dosage of a compound or a combination of compounds depends on several factors, including: the administration method, the type of disease to be treated, the severity of the symptoms, whether administration first occurs at an early or late stage of disease progression, and the age, weight, and health of the patient to be treated.

For combinations that include a synergistic pair of agents identified herein, the recommended dosage for the agent can be less than or equal to the recommended dose as given in the Physician's Desk Reference, 60^{th} Edition (2006).

As described above, the compound(s) in question may be administered orally in the form of tablets, capsules, elixirs or syrups, or rectally in the form of suppositories. Parenteral administration of a compound is suitably performed, for example, in the form of saline solutions or with the compound(s) incorporated into liposomes. In cases where the compound in itself is not sufficiently soluble to be dissolved, a solubilizer such as ethanol can be applied. The correct dosage of a compound can be determined by examining the efficacy of the compound in reporter assays, e.g., one described herein, as well as toxicity in humans.

A therapeutic agent is usually given by the same route of administration that is known to be effective for delivering it as a monotherapy. When used in combination therapy according to the methods of this invention, an agent of Table 1 or an analog thereof is dosed in amounts and frequencies equivalent to or less than those that result in its effective monotherapeutic use if the agent is used monotherapeutically for any indication.

### Additional Applications

If desired, the compounds of the invention may be employed in mechanistic assays to determine whether other combinations, or single agents, are as effective as the combinations of the invention in treating muscular dystrophy (e.g., the types herein) using assays generally known in the art. For example, candidate compounds may be tested, alone or in combination with other agents and applied to cells (e.g., the α7^{+/-}-β-gal mouse myoblast or C2C12 NF-κB-Luciferase cell lines described herein). After a suitable time, reporter gene activity can be measured. Reporter assays such as those described herein can be used to identify additional combination of agents as effective agent for treating muscular dystrophy.

The agents of the invention are also useful tools in elucidating mechanistic information about the biological pathways involved in muscular dystrophy. Such information can lead to the development of new combinations or single agents for treating muscular dystrophy. Methods known in the art to determine biological pathways can be used to determine the pathway, or network of pathways affected by contacting cells (e.g., the α7^{+/-}-β-gal mouse myoblast or C2C12 NF-κB-Luciferase cell lines described herein) with the compounds of the invention. Such methods can include analyzing cellular constituents that are expressed or repressed after contact with the compounds of the invention as compared to untreated, positive or negative control compounds, and/or new single agents and combinations, or analyzing some other activity of the cell such as an enzymatic activity, nutrient uptake, proliferation, or apoptosis. Cellular components analyzed can include gene transcripts, protein expression, and DNA digestion. Suitable methods can include standard biochemistry techniques, radiolabeling the compounds of the invention (e.g., ¹⁴C or ³H labeling), and observing the compounds binding to proteins, e.g., using 2D gels, and gene expression profiling. Once identified, such compounds can be used in in vivo models (e.g., knockout or mutant mice) to further validate the tool or develop new agents or strategies to treat muscular dystrophy.

### Exemplary Candidate Compounds

Peptide Moieties

Peptides, peptide mimetics, and peptide fragments (whether natural, synthetic or chemically modified) are suitable for use in the methods of the invention. Exemplary inhibitors include compounds that reduce the amount of a target protein or RNA levels (e.g., antisense compounds, dsRNA, ribozymes) and compounds that increase the amount of a target protein or RNA levels. Other agents may influence the intraceullar modification or trafficking of a molecule, e.g., NF-κB (e.g., dominant negative proteins or polynucleotides encoding the same).

Antisense Compounds

The biological activity of any protein that increases a symptom of muscular dystrophy, e.g., muscle damage or degeneration, can be reduced through the use of an antisense compound directed to RNA encoding the target protein. Antisense compounds can be identified using standard techniques. For example, accessible regions of the target the mRNA of the target enzyme can be predicted using an RNA secondary structure folding program such as MFOLD (M. Zuker, D. H. Mathews & D. H. Turner, Algorithms and Thermodynamics for RNA Secondary Structure Prediction: A Practical Guide. In: RNA Biochemistry and Biotechnology, J. Barciszewski & B. F. C. Clark, eds., NATO ASI Series, Kluwer Academic Publishers, (1999)). Sub-optimal folds with a free energy value within 5% of the predicted most stable fold of the mRNA are predicted using a window of 200 bases within which a residue can find a complimentary base to form a base pair bond. Open regions that do not form a base pair are summed together with each suboptimal fold and areas that are predicted as open are considered more accessible to the binding to antisense nucleobase oligomers. Other methods for antisense design are described, for example, in U.S. Pat. No. 6,472,521, Antisense Nucleic Acid Drug Dev. 1997 7:439-444, Nucleic Acids Res. 28:2597-2604, 2000, and Nucleic Acids Res. 31:4989-4994, 2003.

RNA Interference

The biological activity of a molecule promoting muscular dystrophy, e.g., NF-κB, can be reduced through the use of RNA interference (RNAi), employing, e.g., a double stranded RNA (dsRNA) or small interfering RNA (siRNA) directed to the signaling molecule in question (see, e.g., Miyamoto et al., Prog. Cell Cycle Res. 5:349-360, 2003; U.S. Pat. Application Publication No. 20030157030). Methods for designing such interfering RNAs are known in the art. For example, software for designing interfering RNA is available from Oligoengine (Seattle, Wash.).

Dominant Negative Proteins

One skilled in the art would know how to make dominant negative proteins to the molecules involved in muscular dystrophy. Such dominant negative proteins are described, for example, in Gupta et al., J. Exp. Med., 186:473-478, 1997; Maegawa et al., J. Biol. Chem. 274:30236-30243, 1999; Woodford-Thomas et al., J. Cell Biol. 117:401-414, 1992).

### EXAMPLES

The following examples are intended to illustrate rather than limit the invention.

### Example 1

### Identification of Therapeutic Agents using a beta-galactosidase Screen Based on Expression of Alpha-7 Integrin

Overexpression of the α7 integrin gene (ITGA7) may suppress symptoms of muscular dystrophy by acting as a compensatory mechanism for stabilizing the sarcolemmal membrane of muscle cells. To identify drug combinations that increase expression of the ITGA7 gene, a screen was performed using α7^{+/-}-β-gal mouse myoblast cells (Flintoff-dye et al., Dev Dyn 234:11-21 (2005)). In this assay, the intensity of a luminescent readout is proportional to the level of the α7-β-galactosidase reporter gene activity.

The α7^{+/-}-β-gal mouse myoblast cells were cultured in T-175 flasks (Corning, Catalog No. 431080) in DMEM growth medium containing high glucose, 10% fetal bovine serum, and 1% penicillin-streptomycin (Cellgrow, Catalog No. 30-002-CI) and passaged at a ratio of 1:10. One T-175 flask of cells provided enough cells to seed five to eight 384-well plates at 10,000 cells/well. Briefly, once approximately 90% confluent, cells were rinsed with 10 mL PBS and 2 mL Trypsin-EDTA was added. The cells were then incubated at room temperature for five minutes. Cell growth medium (8.5 mL) was added to neutralize the trypsin and cells were triturated to break apart clumps. For seeding cells in 384-well assay plates, cell suspensions were combined and cell density was calculated. Additional growth medium was added to dilute the cell suspension to a concentration of 2.5×10⁵ cells per mL, and cells were plated at 10,000 cells in 40 μL per well of a 384-well plate (Matrix Technologies, Custom Order No. BC30316). Compounds were diluted 1:100 in growth media and added at a ratio of 1:10 to each well. The plates were incubated at 37° C. and 5% CO₂ for 72 hours. After incubation, 25 μL Gal-Screen substrate (Applied Biosystems, Catalog No. T1028) was added to each well. Assay plates were incubated at 30° C. and 5% CO₂ for about 2 hours, and luminescence was read on a plate reader.

The fold-stimulation (or induction) of β-galactosidase activity for each combination or compound was calculated by the equation Induction I=ln(T/U) where T was the treated levels and U was the untreated levels. Combination effects were characterized by comparing each data point's Induction to that of the highest single agent combination reference model. The highest single agent model I_{HSA}(C_{X},C_{Y})=max(I_{X},I_{Y}) is a simple reference model where C_{X,Y} are the concentrations of the X and Y compound, and I_{X,Y} are the inductions of the single agents at C_{X,Y}. The Hit Score measurement was used to select hits from the large combination screen. Hit Score H=log f_{X} log f_{Y} Σ max(0,I_{data}) (I_{data}−I_{HSA}) refers to the HSA model. Drug combinations with Hit Scores equal to or greater than 0.6 were selected as efficacious combinations. The data are shown in Table 3.

TABLE 3
Results of α7-βgalactosidase assay

| | | Hit |
|---|---|---|
| Drug 1 | Drug 2 | Score |
| Dipyridamole | MBCQ | 3.362 |
| Everolimus | N-(2-Aminoethyl)-5- | 2.966 |
| | Isoquinolinesulfonamide | |
| Ethaverine hydrochloride | MBCQ | 2.657 |
| EHNA | Everolimus | 2.609 |
| Everolimus | Fasudil | 2.537 |
| Dipyridamole | Everolimus | 2.494 |
| Dilazep dihydrochloride | MBCQ | 2.280 |
| MBCQ | N1 N12-diethylspermine 4HCL | 2.276 |
| Berberine hydrochloride | Papaverine Hydrochloride | 2.248 |
| Fasudil | LY 294002 | 2.150 |
| Berberine hydrochloride | MBCQ | 2.130 |
| Adefovir Dipivoxil | LY 294002 | 2.067 |
| Antimycin A | MBCQ | 2.064 |
| 10-Hydroxycamptothecin | MBCQ | 2.056 |
| Berberine hydrochloride | Fasudil | 2.049 |
| Dipyridamole | S-Petasin | 2.032 |
| Everolimus | MBCQ | 1.935 |
| MS-275 | N-(2-Aminoethyl)-5- | 1.926 |
| | Isoquinolinesulfonamide | |
| Idebenone | Tretinoin | 1.895 |
| 10-Hydroxycamptothecin | Idebenone | 1.843 |
| Adefovir Dipivoxil | MBCQ | 1.841 |
| MBCQ | Simvastatin | 1.813 |
| MBCQ | Suberoylanilide Hydroxamic | 1.796 |
| | Acid | |
| 2-(p-Hydroxyanilino)-4-(p- | Everolimus | 1.796 |
| chlorophenyl) thiazole | | |
| MBCQ | PDTC, NH4 | 1.783 |
| MBCQ | Tretinoin | 1.770 |
| Dilazep dihydrochloride | Everolimus | 1.765 |
| Deguelin | Fasudil | 1.762 |
| LY 294002 | Physostigmine Salicylate | 1.748 |
| 10-Hydroxycamptothecin | LY 294002 | 1.743 |
| Adefovir Dipivoxil | Physostigmine Salicylate | 1.705 |
| Everolimus | Physostigmine Salicylate | 1.670 |
| Ethaverine hydrochloride | Tadalafil | 1.663 |
| Deguelin | MBCQ | 1.644 |
| Deguelin | MS-275 | 1.602 |
| Idebenone | MS-275 | 1.598 |
| EHNA | MBCQ | 1.577 |
| EHNA | Fumagillin | 1.574 |
| Deguelin | N-(2-Aminoethyl)-5- | 1.558 |
| | Isoquinolinesulfonamide | |
| Fasudil | N-(2-Aminoethyl)-5- | 1.553 |
| | Isoquinolinesulfonamide | |
| 10-Hydroxycamptothecin | EHNA | 1.548 |
| MBCQ | Physostigmine Salicylate | 1.524 |
| MBCQ | N-(2-Aminoethyl)-5- | 1.506 |
| | Isoquinolinesulfonamide | |
| MBCQ | S-Petasin | 1.480 |
| MBCQ | Pamidronate Disodium | 1.444 |
| Dipyridamole | Methyldopa | 1.441 |
| Fasudil | N1 N12-diethylspermine 4HCL | 1.440 |
| Everolimus | Tretinoin | 1.440 |
| Florfenicol | MBCQ | 1.435 |
| Fumagillin | N1 N12-diethylspermine 4HCL | 1.430 |
| Fumagillin | N-(2-Aminoethyl)-5- | 1.421 |
| | Isoquinolinesulfonamide | |
| N-(2-Aminoethyl)-5- | N1 N12-diethylspermine 4HCL | 1.413 |
| Isoquinolinesulfonamide | | |
| EHNA | N-(2-Aminoethyl)-5- | 1.408 |
| | Isoquinolinesulfonamide | |
| Physostigmine Salicylate | Tretinoin | 1.395 |
| EHNA | Florfenicol | 1.394 |
| Dipyridamole | Fumagillin | 1.385 |
| Deguelin | Droxidopa | 1.375 |
| Fumagillin | MBCQ | 1.369 |
| Ethaverine hydrochloride | Fasudil | 1.360 |
| Ethaverine hydrochloride | Everolimus | 1.357 |
| 10-Hydroxycamptothecin | Etazolate | 1.354 |
| N-(2-Aminoethyl)-5- | Pamidronate Disodium | 1.346 |
| Isoquinolinesulfonamide | | |
| Deguelin | Simvastatin | 1.341 |
| Deguelin | Everolimus | 1.337 |
| Ergoloid Mesylates | Everolimus | 1.332 |
| Ethaverine hydrochloride | LY 294002 | 1.328 |
| Ethaverine hydrochloride | MS-275 | 1.308 |
| Physostigmine Salicylate | Simvastatin | 1.290 |
| Deguelin | LY 294002 | 1.285 |
| Dilazep dihydrochloride | Ergoloid Mesylates | 1.283 |
| Dipyridamole | MS-275 | 1.267 |
| 10-Hydroxycamptothecin | Dipyridamole | 1.245 |
| Idebenone | MBCQ | 1.244 |
| Berberine hydrochloride | N-(2-Aminoethyl)-5- | 1.244 |
| | Isoquinolinesulfonamide | |
| N1 N12-diethylspermine 4HCL | Pamidronate Disodium | 1.243 |
| EHNA | Pamidronate Disodium | 1.223 |
| Droxidopa | Simvastatin | 1.217 |
| Droxidopa | Tretinoin | 1.216 |
| MS-275 | N1 N12-diethylspermine 4HCL | 1.194 |
| LY 294002 | MBCQ | 1.193 |
| Methyldopa | Tretinoin | 1.184 |
| Donepezil Hydrochloride | Tretinoin | 1.184 |
| Berberine hydrochloride | Florfenicol | 1.180 |
| Dopamine Hydrochloride | MBCQ | 1.178 |
| Levalbuterol Hydrochloride | MBCQ | 1.171 |
| Antimycin A | LY 294002 | 1.159 |
| Dilazep dihydrochloride | MS-275 | 1.158 |
| Alendronate Sodium | Ergoloid Mesylates | 1.150 |
| LY 294002 | N-(2-Aminoethyl)-5- | 1.150 |
| | Isoquinolinesulfonamide | |
| Berberine hydrochloride | Everolimus | 1.131 |
| Ergoloid Mesylates | N-(2-Aminoethyl)-5- | 1.130 |
| | Isoquinolinesulfonamide | |
| Adefovir Dipivoxil | Fasudil | 1.116 |
| Ethaverine hydrochloride | N-(2-Aminoethyl)-5- | 1.092 |
| | Isoquinolinesulfonamide | |
| Adefovir Dipivoxil | Droxidopa | 1.088 |
| Fasudil | Physostigmine Salicylate | 1.087 |
| Ergoloid Mesylates | Pamidronate Disodium | 1.083 |
| Dipyridamole | N-(2-Aminoethyl)-5- | 1.079 |
| | Isoquinolinesulfonamide | |
| Antimycin A | MS-275 | 1.070 |
| EHNA | Idebenone | 1.065 |
| 10-Hydroxycamptothecin | Donepezil Hydrochloride | 1.060 |
| EHNA | S-Petasin | 1.058 |
| Physostigmine Salicylate | S-Petasin | 1.057 |
| Dipyridamole | Florfenicol | 1.039 |
| Berberine hydrochloride | Fumagillin | 1.028 |
| Fasudil | MBCQ | 0.991 |
| Fumagillin | Physostigmine Salicylate | 0.988 |
| 10-Hydroxycamptothecin | Fumagillin | 0.984 |
| Berberine hydrochloride | MS-275 | 0.983 |
| Dipyridamole | Ergoloid Mesylates | 0.978 |
| EHNA | MS-275 | 0.967 |
| Levalbuterol Hydrochloride | Physostigmine Salicylate | 0.967 |
| Adefovir Dipivoxil | Ergoloid Mesylates | 0.964 |
| PDTC, NH4 | Physostigmine Salicylate | 0.964 |
| Alendronate Sodium | N-(2-Aminoethyl)-5- | 0.962 |
| | Isoquinolinesulfonamide | |
| Adefovir Dipivoxil | Fumagillin | 0.942 |
| EHNA | N1 N12-diethylspermine 4HCL | 0.935 |
| Ergoloid Mesylates | Methyldopa | 0.922 |
| Andrographis | Fumagillin | 0.920 |
| Deguelin | Fumagillin | 0.918 |
| EHNA | Ergoloid Mesylates | 0.903 |
| 10-Hydroxycamptothecin | Everolimus | 0.896 |
| Everolimus | Methyldopa | 0.889 |
| Methyldopa | Simvastatin | 0.886 |
| Everolimus | Florfenicol | 0.857 |
| Berberine hydrochloride | Etazolate | 0.855 |
| Droxidopa | Fasudil | 0.854 |
| Adefovir Dipivoxil | Tadalafil | 0.831 |
| Alendronate Sodium | MS-275 | 0.831 |
| Adefovir Dipivoxil | Methyldopa | 0.824 |
| Alendronate Sodium | Idebenone | 0.817 |
| Berberine hydrochloride | S-Petasin | 0.815 |
| Ergoloid Mesylates | Isoetharine Hydrochloride | 0.814 |
| Adefovir Dipivoxil | Ethaverine Hydrochloride | 0.809 |
| Fumagillin | PDTC, NH4 | 0.806 |
| 10-Hydroxycamptothecin | Droxidopa | 0.801 |
| Fasudil | Simvastatin | 0.798 |
| Physostigmine Salicylate | Tadalafil | 0.793 |
| 10-Hydroxycamptothecin | Berberine hydrochloride | 0.782 |
| Methyldopa | N1 N12-diethylspermine 4HCL | 0.758 |
| Florfenicol | S-Petasin | 0.757 |
| EHNA | Physostigmine Salicylate | 0.753 |
| 10-Hydroxycamptothecin | Fasudil | 0.741 |
| MBCQ | Tadalafil | 0.736 |
| Berberine hydrochloride | Ethaverine Hydrochloride | 0.729 |
| EHNA | Methyldopa | 0.725 |
| Berberine hydrochloride | EHNA | 0.719 |
| Droxidopa | MBCQ | 0.696 |
| 10-Hydroxycamptothecin | Pamidronate Disodium | 0.692 |
| Andrographis | MBCQ | 0.692 |
| MS-275 | PDTC, NH4 | 0.686 |
| Berberine hydrochloride | Ergoloid Mesylates | 0.685 |
| 10-Hydroxycamptothecin | Methyldopa | 0.677 |
| 10-Hydroxycamptothecin | Florfenicol | 0.676 |
| MBCQ | Physostigmine Salicylate | 0.669 |
| Fasudil | Methyldopa | 0.663 |

### Example 2

### Identification of Therapeutic Agents by NF-κB Inhibition Screen

NF-κB activation has been positively correlated with muscular dystrophy. To identify drug combinations expected to treat muscular dystrophy by suppressing NF-κB activation, a screen was performed using a cell line engineered to express an NF-κB responsive reporter gene. The cells line, called C2C12 NF-κB-Luciferase (Luc), was derived from mouse myoblast C2C12 cells by chromosomal integration of a construct encoding the luciferase gene and a regulatory element containing 6-copies of the NF-κB response element, a minimal TA promoter, and the TATA box from the thymidine kinase promoter. In this assay, the intensity of luminescence is proportional to the level of reporter gene expression.

C2C12 NF-κB-Luc cells (Panomics, Catalog No. RC0016) cultured in growth medium) were cultured in T-175 flasks or HYPERFlasks (Fisher Scientific) in DMEM growth media containing hygromycin B, 1% penicillin-streptomycin, and 10% fetal bovine serum. Cells were passaged once 90% confluence was achieved at a ratio of approximately 1:8. Briefly, cells were rinsed with PBS (10 mL for a T-175 flask and 50 mL for a HYPERFlask). Trypsin-EDTA was added to the cells (2.5 mL for a T-175 flask and 55 mL for a HYPERFlask), and the cells were incubated at 37° C. and 5% CO₂ for three minutes. Cell growth medium (10 mL for a T-175 flask and 55 mL for a HYPERFlask) was added to neutralize the trypsin and cells were triturated to break apart clumps. For seeding cells in 384-well assay plates, cell suspensions were combined and cell density was calculated. Cells were spun down at 1000 rpm for five minutes and resuspended in Phenol red-free DMEM containing 2 mM L-glutamine, hygromycin B, 1% penicillin-streptomycin, and 10% fetal bovine serum. Additional assay medium was added to dilute the cell suspension to a concentration of 2.5×10⁵ cells per mL. Cells were plated at 10,000 cells in 40 μL per well of a 384-well plate (Matrix Technologies, Custom Order No. BC30316), and incubated at 37° C. and 5% CO₂ for 24 hours. On the following day, compounds were diluted 1:100 in assay medium containing 40 ng/mL TNFα. The compounds and TNFα (4 ng/mL final concentration) were then simultaneously added at a ratio of 1:10 to each well. The plates were incubated at 37° C. and 5% CO₂ for an additional 18 hours. On the following day, the plates were brought to room temperature for 20 minutes before adding 40 μL of SteadyLite reagent (Perkin Elmer, Catalog No. 6016989) to each well. After incubation for 15 minutes at room temperature, luminescence was read on a plate reader.

Combination effects were characterized by comparing each data point's Inhibition (I=1−T/U where T is treated levels and U is untreated levels) to that of the Loewe additivity combination reference model that was derived from the single agent curves. Loewe additivity, where I_{Loewe} (Cₓ, C_{y}) is the inhibition that satisfies (Cₓ/ECₓ)+(C_{Y}/EC_{Y})=1. Here C_{X,Y} are the concentrations of the X and Y compound, and EC_{X,Y} are the effective concentrations at I_{Loewe} for the single agent curves. Loewe additivity is a generally accepted reference for synergy, as it represents the combination response generated if X and Y are the same compound. The Synergy Score measurement was used to select hits from the large combination screen. Synergy Score S=log fₓ log f_{y} Σmax(0,I_{data}) (I_{data}−I_{Loewe}) is the positive-gated, inhibition-weighted volume over Loewe additivity. A Synergy Score of equal to or greater than 1 indicated a synergistic effect of the drug combination on NF-κB inhibition. The results are shown in Table 4.

TABLE 4
Results of NF-κB inhibition screen

| | | Synergy |
|---|---|---|
| Drug 1 | Drug 2 | Score |
| Dilazep dihydrochloride | Dexamethasone | 4.378 |
| Etonogestrel | Dilazep | 3.163 |
| Prednisolone | Dilazep dihydrochloride | 4.434 |
| Prednisolone | Ergoloid Mesylates | 3.019 |
| Mitoxantrone Hydrochloride | Etonogestrel | 2.742 |
| Prednisolone | Ethaverine Hydrochloride | 2.947 |
| Prednisolone | Dihydroergotamine Mesylate | 3.481 |
| Trequinsin Hydrochloride | Mitoxantrone Hydrochloride | 3.304 |
| NKH 477 | Ethaverine Hydrochloride | 1.691 |
| Ergoloid Mesylates | Dexamethasone | 3.989 |
| Tretinoin | Etonogestrel | 2.458 |
| Prednisolone | Bromocriptine Mesylate | 2.354 |
| NKH 477 | Mitoxantrone Hydrochloride | 3.036 |
| Prednisolone | Cilobradine Hydrochloride | 2.226 |
| Verapamil Hydrochloride | Prednisolone | 4.039 |
| MS-275 | Mitoxantrone Hydrochloride | 0.931 |
| Mitoxantrone Hydrochloride | Ethaverine Hydrochloride | 3.185 |
| MS-275 | Dilazep | 2.142 |
| Prednisolone | Dilazep | 2.099 |
| Verapamil Hydrochloride | Mitoxantrone Hydrochloride | 2.452 |
| Trequinsin Hydrochloride | Dexamethasone | 3.594 |
| Etonogestrel | Calcitriol | 1.977 |
| NKH 477 | MS-275 | 3.053 |
| Ethaverine Hydrochloride | Dexamethasone | 2.665 |
| NKH 477 | Etonogestrel | 1.890 |
| Mitoxantrone Hydrochloride | Bromocriptine Mesylate | 1.869 |
| Mitoxantrone Hydrochloride | Dilazep dihydrochloride | 3.241 |
| Prednisolone | Drotaverine Hydrochloride | 4.114 |
| Mitoxantrone Hydrochloride | Dihydroergotamine Mesylate | 1.481 |
| Etonogestrel | Dihydroergotamine Mesylate | 1.791 |
| Mitoxantrone Hydrochloride | Calcitriol | 2.606 |
| Mivacurium Chloride | Mitoxantrone Hydrochloride | 1.727 |
| Etonogestrel | Demecarium Bromide | 1.682 |
| Prednisolone | NKH 477 | 2.678 |
| Mitoxantrone Hydrochloride | Cilobradine Hydrochloride | 1.650 |
| Mivacurium Chloride | Etonogestrel | 1.647 |
| Prednisolone | Calcitriol | 2.589 |
| Mitoxantrone Hydrochloride | Deflazacort | 0.736 |
| Trequinsin Hydrochloride | Prednisolone | 2.600 |
| Sulforaphane | Dihydroergotamine Mesylate | 1.618 |
| Trequinsin Hydrochloride | Dilazep | 1.603 |
| Verapamil Hydrochloride | Dexamethasone | 3.617 |
| Mitoxantrone Hydrochloride | Ergoloid Mesylates | 2.261 |
| Prednisolone | MS-275 | 2.837 |
| Dilazep | Dexamethasone | 1.532 |
| Dihydroergotamine Mesylate | Dexamethasone | 3.447 |
| Prednisolone | Epiandrosterone | 1.499 |
| Etonogestrel | Dilazep dihydrochloride | 1.477 |
| Etonogestrel | Ethaverine Hydrochloride | 1.468 |
| Procaterol Hydrochloride | Etonogestrel | 1.467 |
| Prednisolone | Mitoxantrone Hydrochloride | 1.736 |
| Trequinsin Hydrochloride | MS-275 | 1.445 |
| Sulforaphane | Mitoxantrone Hydrochloride | 1.437 |
| Trequinsin Hydrochloride | NKH 477 | 1.404 |
| MBCQ | Etonogestrel | 1.382 |
| Drotaverine Hydrochloride | Dexamethasone | 4.146 |
| Prednisolone | Mivacurium Chloride | 1.321 |
| Efavirenz | Dilazep dihydrochloride | 1.318 |
| Mitoxantrone Hydrochloride | MBCQ | 1.059 |
| Levalbuterol Hydrochloride | Dilazep | 1.310 |
| Unithiol Monohydrate | Etonogestrel | 1.310 |
| Verapamil Hydrochloride | Etonogestrel | 1.279 |
| MS-275 | Deflazacort | 1.274 |
| Vinburnine | Mitoxantrone Hydrochloride | 1.273 |
| Vitamin A Acetate | Mitoxantrone Hydrochloride | 1.272 |
| Prednisolone | N-Methyl-Paroxetine | 1.260 |
| NKH 477 | Dilazep dihydrochloride | 1.248 |
| Tretinoin | MS-275 | 1.247 |
| Mitoxantrone Hydrochloride | Dexamethasone | 1.458 |
| Dexamethasone | Bromocriptine Mesylate | 1.242 |
| Procaterol Hydrochloride | Mivacurium Chloride | 1.240 |
| Trequinsin Hydrochloride | Deflazacort | 1.800 |
| Prednisolone | Amoxapine | 1.232 |
| Etonogestrel | Bromocriptine Mesylate | 1.206 |
| NKH 477 | Ergoloid Mesylates | 1.196 |
| Dilazep | Dihydroergotamine Mesylate | 1.185 |
| Mitoxantrone Hydrochloride | Demecarium Bromide | 1.183 |
| NKH 477 | MBCQ | 1.175 |
| Epiandrosterone | Dexamethasone | 1.169 |
| MBCQ | Ethaverine Hydrochloride | 0.894 |
| Rosuvastatin calcium | Mitoxantrone Hydrochloride | 1.161 |
| Quinidine | Prednisolone | 1.158 |
| N-Methyl-Paroxetine | Mitoxantrone Hydrochloride | 1.149 |
| Bromocriptine Mesylate | Bethanechol Chloride | 1.146 |
| Prednisolone | MBCQ | 2.450 |
| Dexamethasone | Cilobradine Hydrochloride | 1.137 |
| Otilonium Bromide | MBCQ | 1.135 |
| Mitoxantrone Hydrochloride | Drotaverine Hydrochloride | 1.483 |
| Drotaverine Hydrochloride | Calcitriol | 2.252 |
| Dilazep dihydrochloride | Demecarium Bromide | 1.126 |
| Prednisolone | Piperacetazine | 1.121 |
| MS-275 | Dexamethasone | 1.120 |
| Trequinsin Hydrochloride | Mivacurium Chloride | 1.120 |
| NKH 477 | Calcitriol | 1.115 |
| NKH 477 | Mivacurium Chloride | 1.112 |
| Etonogestrel | Bexarotene | 1.101 |
| Salmeterol Xinafoate | MS-275 | 1.083 |
| MS-275 | Etonogestrel | 1.081 |
| Vitamin A Acetate | Trequinsin Hydrochloride | 1.077 |
| Otilonium Bromide | Etonogestrel | 1.074 |
| Verapamil Hydrochloride | NKH 477 | 1.057 |
| MS-275 | MBCQ | 1.055 |
| Demecarium Bromide | Calcitriol | 1.044 |
| Mitoxantrone Hydrochloride | Methoxsalen | 1.043 |
| NKH 477 | Dilazep | 1.041 |
| Vinburnine | Prednisolone | 1.039 |
| Dihydroergotamine Mesylate | Calcitriol | 1.038 |
| MS-275 | Ethaverine Hydrochloride | 1.035 |
| K-252a | Ethaverine Hydrochloride | 1.033 |
| Mivacurium Chloride | K-252a | 1.031 |
| Procaterol Hydrochloride | Dilazep dihydrochloride | 1.015 |
| Tretinoin | Ethaverine Hydrochloride | 1.015 |
| Mivacurium Chloride | Calcitriol | 1.008 |
| NKH 477 | Deflazacort | 1.005 |
| Ergoloid Mesylates | Calcitriol | 1.003 |
| MS-275 | Dilazep dihydrochloride | 1.299 |
| Dilazep | Calcitriol | 0.998 |
| MBCQ | Deflazacort | 2.159 |
| Dihydroergotamine Mesylate | Deflazacort | 1.203 |
| Dilazep | Deflazacort | 0.980 |
| Ergoloid Mesylates | Deflazacort | 1.417 |
| Ethaverine Hydrochloride | Deflazacort | 1.556 |
| MBCQ | Dilazep dihydrochloride | 0.760 |
| Trequinsin Hydrochloride | Dilazep dihydrochloride | 0.732 |
| Dilazep dihydrochloride | Deflazacort | 1.330 |
| Drotaverine Hydrochloride | Deflazacort | 2.267 |
| Dipyridamole | Dexamethasone | 1.993 |
| Prednisolone | Dipyridamole | 3.016 |
| Dipyridamole | Deflazacort | 0.975 |
| Papaverine Hydrochloride | Prednisolone | 2.980 |
| Dexamethasone | Papaverine Hydrochloride | 2.762 |
| AL-438 | Dilazep dihydrochloride | 2.511 |
| AL-438 | Papaverine Hydrochloride | 2.216 |
| AL-438 | Ergoloid Mesylates | 2.171 |
| Dexamethasone | Zardaverine | 1.953 |
| AL-438 | Dihydroergotamine Mesylate | 1.881 |
| Deflazacort | Zardaverine | 1.650 |
| Prednisolone | Zardaverine | 1.430 |
| Deflazacort | Papaverine Hydrochloride | 1.335 |
| 2-(4-acetoxyphenyl)-2-chloro-N- | Dilazep dihydrochloride | 1.183 |
| methyl-ethylammonium chloride | | |
| Dipyridamole | Mitoxantrone Hydrochloride | 1.307 |
| AL-438 | Ethaverine Hydrochloride | 1.215 |
| Mitoxantrone Hydrochloride | Papaverine Hydrochloride | 1.173 |
| 2-(4-acetoxyphenyl)-2-chloro-N- | Ergoloid Mesylates | 0.966 |
| methyl-ethylammonium chloride | | |
| 2-(4-acetoxyphenyl)-2-chloro-N- | Papaverine Hydrochloride | 0.775 |
| methyl-ethylammonium chloride | | |
| Mitoxantrone Hydrochloride | Tetrahydropapaveroline | 0.694 |
| | Hydrobromide | |
| Dexamethasone | Tetrahydropapaveroline | 0.673 |
| | Hydrobromide | |
| Prednisolone | Tetrahydropapaveroline | 0.593 |
| | Hydrobromide | |
| Deflazacort | Tetrahydropapaveroline | 0.353 |
| | Hydrobromide | |
| 2-(4-acetoxyphenyl)-2-chloro-N- | Ethaverine Hydrochloride | 0.805 |
| methyl-ethylammonium chloride | | |
| 2-(4-acetoxyphenyl)-2-chloro-N- | Dihydroergotamine Mesylate | 0.911 |
| methyl-ethylammonium chloride | | |

### Other Embodiments

All publications, patent applications, and patents mentioned in this specification are herein incorporated by reference.

Various modifications and variations of the described compositions, methods, and kits of the invention will be apparent to those skilled in the art without departing from the scope and spirit of the invention. Although the invention has been described in connection with specific embodiments, it will be understood that the invention as claimed should not be unduly limited to such specific embodiments. Indeed, various modifications of the described modes for carrying out the invention that are obvious to those skilled in the fields of molecular biology, medicine, immunology, pharmacology, cell biology, or related fields are intended to be within the scope of the invention.

What is claimed is:

1. A method for treating a patient having muscular dystrophy, said method comprising administering to said patient an effective amount of a pair of agents selected from the pairs of Table 1 or analogs thereof.

2. The method of claim 1, wherein said muscular dystrophy is Duchenne muscular dystrophy.

3. The method of claim 1, wherein said agents are a pair selected from the pairs of Table 1.

4. The method of claim 1, further comprising administering a third agent that is a corticosteroid.

5. The method of claim 1, wherein said agents are administered within 28 days of each other.

6. The method of claim 5, wherein said agents are administered within 10 days of each other.

7. The method of claim 6, wherein said agents are administered within 3 days of each other.

8. The method of claim 7, wherein said agents are administered within 24 hours of each other.

9. The method of claim 8, wherein said agents are administered within 1 hour of each other or substantially simultaneously.

10. The method of claim 1, wherein at least one of said pair of agents is administered orally, parenterally, systemically, topically, or inhalationally.

11. The method of claim 1, wherein said patient is a human.

12. A composition comprising a pair of agents selected from the pairs of Table 1 or analogs thereof.

13. The composition of claim 12, wherein said agents or analogs thereof are present in amounts that, when administered together to a patient having muscular dystrophy, are effective to treat said patient.

14. The composition of claim 12, wherein said muscular dystrophy is Duchenne muscular dystrophy.

15. The composition of claim 12, wherein said agents are a pair selected from the pairs of Table 1.

16. The composition of claim 16, wherein said composition is formulated for oral, parenteral, systemic, topical, or inhalational administration.

17. The composition of claim 12, wherein said composition consists of active ingredients and excipients and said active ingredients consist of said pair of agents or analogs thereof.

18. A kit comprising:
(a) a pair of agents selected from the pairs of Table 1 or analogs thereof; and
(b) instructions for administering said pair of agents to a patient having muscular dystrophy.

19. The kit of claim 18, wherein said muscular dystrophy is Duchenne muscular dystrophy.

20. The kit of claim 18, wherein said pair of agents is a pair selected from the pairs of Table 1.

21. The kit of claim 18, wherein said kit comprises a composition comprising said pair of agents.

22. The kit of claim 18, wherein said agents are formulated separately.

23. The kit of claim 18, wherein at least one of said agents is formulated for oral, parenteral, systemic, topical, or inhalational administration.

24. A kit comprising:
(a) a first agent of a pair of agents selected from the pairs of Table 1 or analogs thereof; and
(b) instructions for administering said first agent with the second agent of said pair of agents to a patient having muscular dystrophy.

25. The kit of claim 24, wherein said muscular dystrophy is Duchenne muscular dystrophy.

26. The kit of claim 24, wherein said pair of agents is a pair selected from the pairs of Table 1.

27. The kit of claim 24, wherein at least one of said pair of agents is formulated for oral, parenteral, systemic, topical, or inhalational administration.
